(19) United States
(12) Patent Application Publication
(10) Pub. No.: US 2020/0237278 A1
(43) Pub. Date: Jul. 30, 2020

(54) INTRAVENOUS CATHETER AND BLOOD COLLECTION DEVICE

(71) Applicant: MMI, LLC, San Diego, CA (US)

(72) Inventor: Hooman Asbaghi, San Diego, CA (US)

(73) Assignee: MMI, LLC, San Diego, CA (US)

(21) Appl. No.: 16/633,899

(22) PCT Filed: Jul. 27, 2018
(86) PCT No.: PCT/US18/44125

§ 371 Date: Jan. 24, 2020

Related U.S. Application Data
(60) Provisional application No. 62/538,381, filed on Jul. 28, 2017.

(51) Int. Cl. A61B 5/15

(52) U.S. Cl. CPC A61B 5/15003; A61B 5/15074; A61B 5/150389; A61B 5/150213; A61B 5/150885

(57) ABSTRACT

A device that permits collection of blood from a patient by a needle and also places a catheter tube in the circulatory system of the patient.

### CROSS REFERENCE TO RELATED APPLICATION

This application claims the benefit of priority to U.S. Provisional Patent Application Ser. No. 62/538,381, filed Jul. 28, 2017, incorporated herein by reference.

### FILED OF THE INVENTION

Embodiments of the present invention relate, in general, to medical catheters and devices for collecting blood, and, in particular, to such combinations that include needles that retract after use and provisions for attachment to a blood collection vial.

### BACKGROUND OF THE INVENTION

Fluid access into the vasculature of a patient may be necessary, or desirable, for any of several different reasons. When such access is desirable, a fluid flow path is generally established between an extracorporeal fluid source and the vasculature. Moreover, when an infusion protocol is involved that requires periodic injections, an established fluid access site that can be repetitively used for a sequence of different injections may be required. Establishing such an access site, however, can be problematic. Further, obtaining a sample of the patient's blood is performed separately from any infusion, and typically the blood collection and infusion require separate penetrations of the patient's skin.

Various embodiments of the inventions shown herein provide for a single penetration that not only provides the blood sample, but also establishes an infusion access site.

### SUMMARY OF THE INVENTION

Various embodiments of the present invention pertain to improved method and apparatus for using a single needle stick on a patient to both obtain a blood sample, and also to insert a catheter into the circulatory system of the patient.

Various embodiments of the present invention include a single device that includes a retractable needle that is in fluid communication by way of a fitting suitable for an evacuated blood collection vial, and a catheter having a flexible lumen that surrounds the needle when it is in the non-retracted, ready to use configuration.

Various embodiments of the present invention include a variable length fluid conduit from the needle assembly to the blood vial collection fitting. In some embodiments this variable length fluid communication is accomplished by stretching or compressing a flexible tube. In yet other embodiments it is accomplished by a tube that has a compacted, stored length that is greater than the extended length. In still further embodiments the fluid conduit has a substantially fixed length, and the conduit slides and advances in a proximal direction from the proximal end of the device when the needle is retracted.

Various embodiments of the invention include means for retaining the sliding needle body in the extended position, but permitting the needle body to retract with the catheter assembly is removed from contact with the needle assembly. In some embodiments this is accomplished by establishing a friction fit between the sliding needle body and the housing of the device.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

The accompanying drawings incorporated in and forming a part of the specification illustrate several aspects of the present disclosure, and together with the description serve to explain the principles of the invention; it being understood, however, that the described embodiments are not limited to the precise arrangements shown. In the drawings, a first set of reference numerals are used in FIGS. 1-27, and a second set of reference numerals are used in FIGS. 28-38.

FIG. 1 is a side view of one version of a safety catheter shown with a shield engaged with a holder of the safety catheter.

FIG. 2 is an exploded perspective view of the safety catheter of FIG. 1 having a luer assembly, a shuttle assembly, and a holder assembly.

FIG. 3 is a side cross-section view of the luer assembly of the safety catheter shown in FIG. 2.

FIG. 4 is a side cross-section view of the shuttle assembly of the safety catheter shown in FIG. 2.

FIG. 5 is a side cross-section view of the holder assembly of the safety catheter shown in FIG. 2.

FIG. 6 is a perspective view of the shield shown in FIG. 2.

FIG. 7 is a perspective view of the holder shown in FIG. 2.

FIG. 8 is a perspective view of the luer or hollow outer portion shown in FIG. 2.

FIG. 9 is a perspective view of the body top shown in FIG. 2.

FIG. 10 is a perspective view of the shuttle body shown in FIG. 2.

FIG. 11 is a side view of the stylet shown in FIG. 2.

FIG. 12 is a perspective view of the eyelet shown in FIG. 2.

FIG. 13 is a perspective view of the spring shown in FIG. 2.

FIG. 14 is a side view of the catheter shown in FIG. 2.

FIG. 15 is a perspective view of the one-way valve shown in FIG. 2.

FIG. 16 is a perspective view of the filter or stop shown in FIG. 2.

FIG. 17 is a perspective view of the actuator shown in FIG. 2.

FIG. 18 is a side cross-section view of the safety catheter of FIG. 1 shown in a pre-use configuration with the shield in place.

FIG. 19 is a side cross-section view of the safety catheter of FIG. 18, shown rotated ninety degrees.

FIG. 19A is a side view of the safety catheter of FIG. 19.

FIG. 20 is a side cross-section view of the safety catheter of FIG. 1, shown with the shield removed in a configuration for accessing the vasculature of a patient.

FIG. 21 is a side cross-section view of the safety catheter of FIG. 20, shown rotated ninety degrees.

FIG. 21A is a side view of the safety catheter of FIG. 20.

FIG. 22 is a side cross-section view of the safety catheter of FIG. 1, shown with the actuator and the luer assembly distally advanced.

FIG. 23 is a side cross-section view of the safety catheter of FIG. 22, shown rotated ninety degrees.

FIG. 23A is a side view of the safety catheter of FIG. 23.

FIG. 24 is a side cross-section view of the safety catheter of FIG. 1, shown with the luer assembly disengaged from the shuttle assembly and handle assembly.

FIG. 25 is a side cross-section view of the safety catheter of FIG. 24, shown rotated ninety degrees.

FIG. 25A is a side view of the safety catheter of FIG. 25.

FIG. 26 is a side cross-section view of the safety catheter of FIG. 1, shown with the shuttle assembly retracted into the handle assembly.

FIG. 27 is a side cross-section view of the safety catheter of FIG. 26, shown rotated ninety degrees.

FIG. 27A is side view of the safety catheter of FIG. 27.

FIG. 28 is a side, partially cross sectional, schematic representation of a combined blood collection device and intravenous catheter according to one embodiment of the present invention in the fully extended position.

FIG. 29 is a side, partially cross sectional, schematic representation of the needle assembly of FIG. 28.

FIG. 30 is a side, partially cross sectional schematic representation from above of the catheter of FIG. 28.

FIG. 31 is a side, partially cross sectional schematic representation of a portion of the assembly of FIG. 28.

FIG. 32 is a view of the apparatus of FIG. 28 with the needle in the fully extended position and with the catheter and adapter removed for the sake of clarity.

FIG. 33 is a view of the apparatus of FIG. 32 with the needle shown in the fully retracted position.

FIG. 34 is a side, partially cross sectional, schematic representation of a blood collection and catheter device according to another embodiment of the present invention, with the needle shown in the extended position.

FIG. 35 is a view of the apparatus of FIG. 34 with the needle shown in the fully retracted position.

FIG. 36 is a side, partially cross sectional, schematic representation of a combined blood collection device and intravenous catheter according to yet another embodiment of the present invention.

FIG. 37 is a side, partially cross sectional, schematic representation of a blood collection and catheter device according to the device shown in FIG. 36, with several components removed for the sake of clarity, with the needle shown in the extended position.

FIG. 38 is a view of the apparatus of FIG. 37 with the needle shown in the fully retracted position.

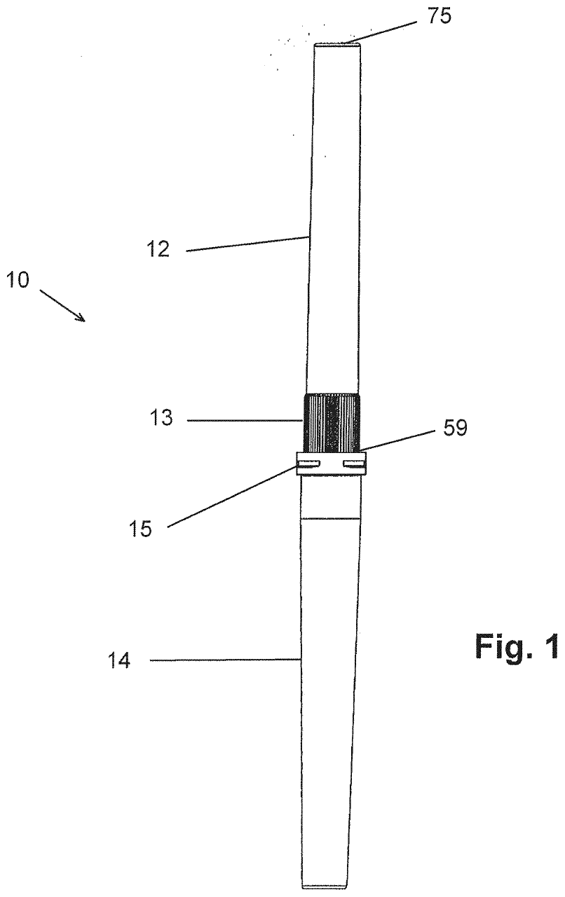

### ELEMENT NUMBERING—FIGS. 1-27

The following is a list of element numbers and at least one noun used to describe that element. It is understood that none of the embodiments disclosed herein are limited to these nouns, and these element numbers can further include other words that would be understood by a person of ordinary skill reading and reviewing this disclosure in its entirety.

| | |
|---|---|
| 10 | catheter |
| 11 | |
| 12 | handle |
| 13 | textured surface |
| 14 | cap; shield |
| 15 | flats |
| 16 | luer assembly |
| 17 | projections |
| 20 | body portion |
| 22 | cannula |
| 24 | eyelet |
| 26 | one-way valve |
| 28 | guides |
| 30 | proximal end |
| 32 | distal end |
| 33 | proximal end |
| 34 | distal end |
| 35 | textured surface |
| 36 | stylet |
| 38 | shuttle body |
| 40 | plug |
| 42 | distal tip |
| 44 | proximal end |
| 45 | proximal end |
| 46 | distal end or proximal end |
| 47 | channel |
| 48 | indents or flats |
| 49 | cavity |
| 50 | shuttle body assembly |
| 52 | handle body |
| 54 | body top |
| 58 | proximal end |
| 59 | distal end |
| 61 | annular flange |
| 62 | flash window |
| 63 | handle assembly |
| 65 | outer portion |
| 66 | resilient fingers |
| 67 | inner portion |
| 68 | projections |
| 69 | distal end |
| 70 | spring |
| 71 | stops |
| 72 | annular flange |
| 73 | gaps |
| 75 | proximal end |
| 77 | internal chamber |
| 80 | actuator |
| 81 | lateral flanges |
| 90 | flat |
| 92 | annular collar |
| 94 | distal valve portion |
| 95 | base |
| 96 | slit |
| 97 | dorsal thumb pad |
| 98 | proximal end |
| 100 | distal end |
| 102 | rails |
| 104 | lateral arms |
| 106 | distal retention latches |
| 108 | lateral projections |
| 110 | proximal living hinges |
| 112 | bands |
| 115 | conical bevel |

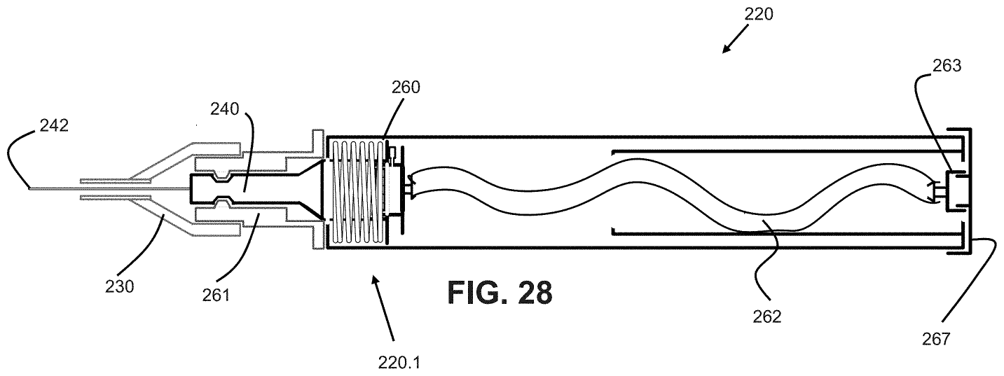

### ELEMENT NUMBERING—FIGS. 28-38

The following is a list of element numbers and at least one noun used to describe that element. It is understood that none of the embodiments disclosed herein are limited to these nouns, and these element numbers can further include other words that would be understood by a person of ordinary skill reading and reviewing this disclosure in its entirety.

| | |
|---|---|
| 220, 320, 420 | apparatus, device |
| .1 | extended; prior to use |
| .2 | extended; after use |
| .3 | retracted; after use |
| 30 | catheter assembly |
| 31 | body |
| .1 | inner surface |
| 32 | flexible lumen; tube |
| .2 | fluid path |
| 33 | luer fitting |
| 36 | wings |
| 40 | needle assembly |
| 41 | body |
| .1 | notches; means for axial restraint; |
| | means for limiting retraction |
| 42 | cannula; first needle |
| .1 | fluid path |
| 43 | tubing connector |
| 45 | casing |
| .1 | filter |
| .2 | one way valve; vent |
| .3 | spring shoulder; spring travel stop |
| .4 | travel stop; abutment |
| 46 | sheath |
| 60 | housing assembly |
| 61 | adaptor |
| .1 | projections; means for axial restraint; |
| | means for limiting retraction |
| .2 | flexible arms |
| .3 | proximal flange |
| .4 | outer surface |
| 62 | flexible tube |
| .1 | fluid path |
| .2 | needle body connection |
| .3 | collection vial connection |
| .4 | storage |
| .5 | after usage |
| .6 | compressed |
| .7 | free state |
| .8 | extended |
| 63 | luer fitting; vial collection fitting |
| 64 | cannula; second needle |
| 65 | protective casing |
| .1 | first compartment |
| .2 | second compartment |
| .3 | travel stop; abutment |
| .4 | distal face |
| 66 | spring |
| .1 | compressed |
| .2 | released |
| 67 | end cap |

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “preferably,” refers to features and aspects that are present in at least one embodiment, but which are optional for some embodiments, it therefore being understood that use of the word “preferably” implies the term “optional.”

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

Various references may be made to one or more methods of manufacturing. It is understood that these are by way of example only, and various embodiments of the invention can be fabricated in a wide variety of ways, such as by casting, sintering, sputtering, welding, electrodischarge machining, milling, as examples. Further, various other embodiment may be fabricated by any of the various additive manufacturing methods, some of which are referred to 3-D printing.

This document may use different words to describe the same element number, or to refer to an element number in a specific family of features. It is understood that such multiple usage is not intended to provide a redefinition of any language herein. It is understood that such words demonstrate that the particular feature can be considered in various linguistical ways, such ways not necessarily being additive or exclusive.

As shown in the figures, versions illustrated herein may useful as a device for manipulating a stylet and/or any other stiffening or penetration element to position a catheter in fluid communication with the vasculature of a patient, and for subsequently concealing the stylet to prevent inadvertent “sticks” with the stylet. In one version, the retraction of the stylet or needle cannula is performed in a controlled manner, where controlled retraction may limit or mitigate tissue damage that can be associated with uncontrolled retraction that occurs automatically without input from a clinician.

When a catheter is used to establish a fluid access site into the vasculature of a patient, the catheter is generally flexible. Once positioned, a flexible cannula may be beneficial in reducing patient discomfort and in minimizing tissue damage. The flexible catheter, however, may need to be stiffened for insertion so that the distal end of the catheter, or cannula, can be passed through tissue and positioned in the vasculature. This stiffening can be accomplished, for example, by using a stylet that can be selectively inserted into the lumen of the catheter to stiffen the catheter during insertion. After the stiffened catheter has been properly positioned in the vasculature, the stylet can be removed from the catheter to leave the flexible catheter in fluid communication with the vasculature for delivery or removal of fluid therefrom.

Versions of the safety catheter described herein provide for a stylet or needle cannula that is passively retracted from a flexible catheter after the flexible catheter is properly positioned. Passively retracting the stylet after positioning the catheter may reduce the risk of accidental needle sticks by safely securing the stylet upon completion of the catheter insertion. In at least one version, a passive release refers to automatically releasing a needle or shuttle assembly for retraction. However, it will be appreciated that upon passive release, where a needle assembly is free to pass into a secured position, a user may still control the timing of the actual release to provide controlled retraction. Versions herein provide for the controlled retraction of the stylet after positioning the flexible catheter, where controlled retraction may allow the stylet to be safely secured without causing tissue damage that may be associated with an abrupt or uncontrolled retraction.

Versions described herein are directed to a catheter device and system that can be positioned to establish a single fluid access site for multiple infusions of a fluid medicament into the vasculature. The safety catheter system may be configured with a single-step operation such that the flexible catheter is separated from the stylet in an automated manner and the stylet is concealed after placement of the catheter to prevent accidental needle sticks and can include an actuator and/or other release device, mechanism, or component to facilitate controlled retraction.

FIGS. 1-27 are prior art, as patented in U.S. Pat. No. 8,216,188, issued Jul. 10, 2012.

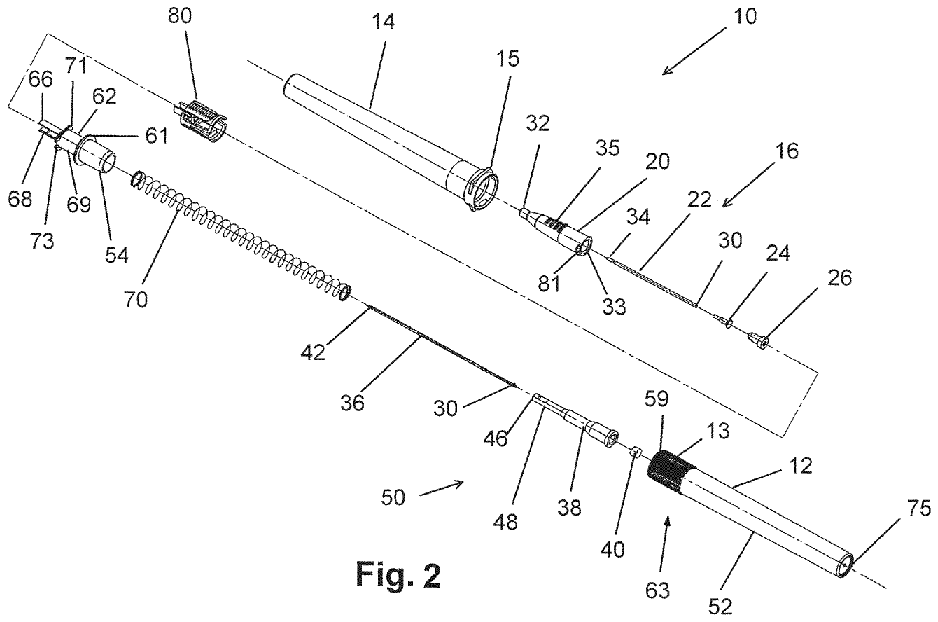

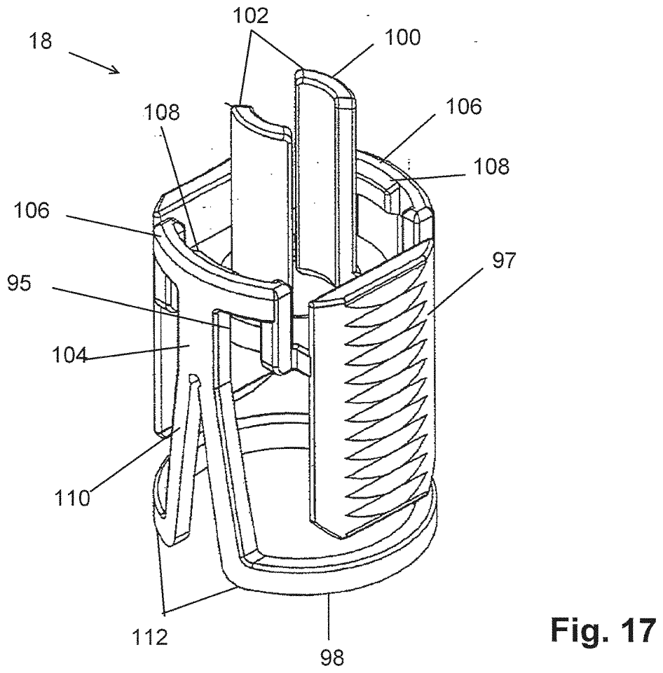

Referring to FIGS. 1 and 2, one version of a safety catheter 10 is provided that is configured for insertion into the vasculature of a patient. The safety catheter 10 may be used to establish a single fluid access site into the vasculature of the patient that can be repetitively or sequentially used by extracorporeal fluid sources such as, but not limited to, a hypodermic syringe or IV pump (not shown). Generally, versions of the safety catheter 10 are configured to stiffen a flexible catheter or cannula 22 for insertion into the vasculature of a patient. Once the cannula 22 is properly positioned, the safety catheter 10 is configured to passively and/or automatically release a stylet 36, or any other suitable stiffening and/or penetration element, for withdrawal from the catheter. In one version, upon passive release of the stylet 36 from the cannula 22, the stylet 36 is configured for controlled retraction via an actuator 80 (FIG. 17) into a handle body 52 such that the sharp distal tip 42 of the stylet 36 is concealed to prevent accidental needle sticks. Controlled retraction of the stylet 36, after passive release from the cannula 22, may reduce or prevent tissue damage associated with an uncontrolled or abrupt retraction.

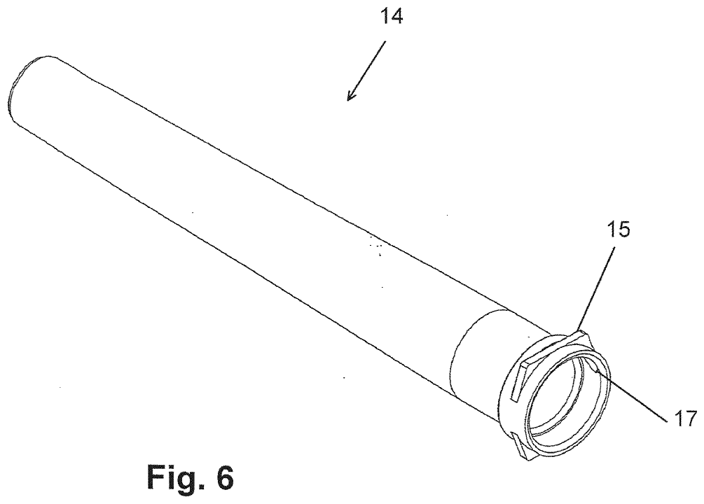

Referring to FIG. 1, one version of a safety catheter 10 is shown in a pre-deployment or pre-use configuration. More specifically, the safety catheter 10 is shown having a handle 12 with a textured surface 13 and a cap or shield 14. As illustrated, the shield 14 is engaged with the handle 12 to prevent exposure and contamination of the safety catheter 10. The shield 14 may have any suitable configuration designed to prevent exposure of the cannula 22 and stylet 36 (FIG. 2). Shield 14 comprises any suitable shape or grip and may be transparent or semitransparent to facilitate visualization of the cannula 22 and stylet 36. Referring to FIG. 6, the shield 14 may include flats 15 to prevent the safety catheter 10 from rolling on a flat surface and to facilitate automated engagement with the handle 12. The shield 14 may further include one or a plurality of projections 17, or any other suitable coupling, configured to engage the handle 12 to provide a secure coupling. The handle 12 may include any suitable corresponding coupling means.

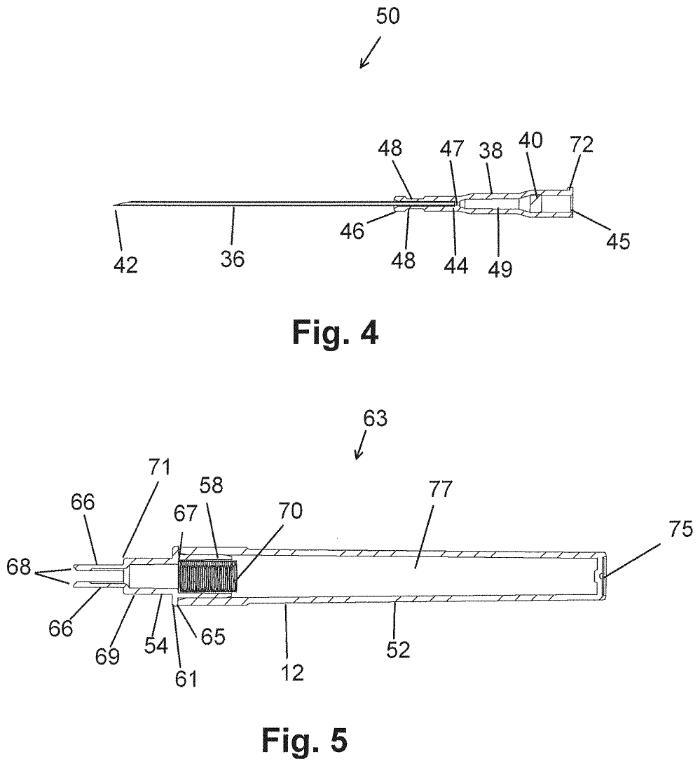

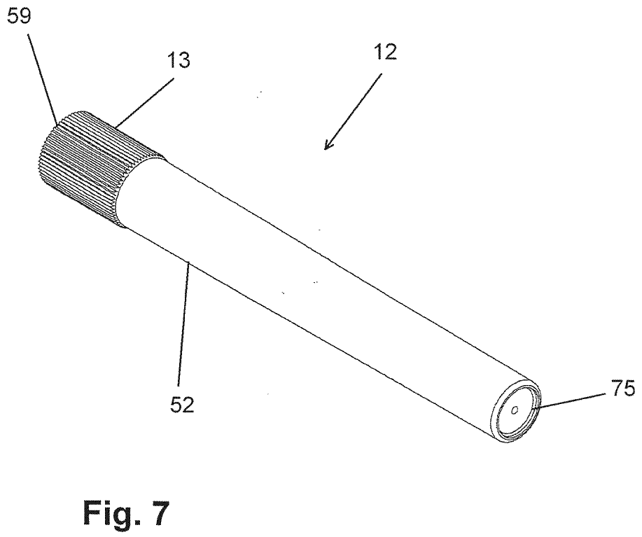

Referring to FIG. 7, the handle 12 may have any suitable gripping surface, such as textured surface 13, to facilitate handling, separation from the shield, operation, or the like. The handle 12 of the safety catheter, in one version, includes an elongated handle body 52 that has a proximal end 75 and a distal end 59. It is also formed with an internal chamber 77. During operation, after a catheter has been inserted into the vasculature of a patient, the handle body 52 is configured to retain the shuttle assembly 50 (FIG. 4) upon retraction of the stylet. The chamber 77 serves as a compartment for the stylet 36 to prevent accidental needle sticks and to prevent re-use.

In FIG. 1, the shield 14 is shown engaged with the handle 12, where any suitable coupling between the shield 14 and the handle, including a friction fit, a snap fit, a threaded fit, shrink wrap, tamper evident packaging, or the like, is contemplated. In one version, once the shield 14 is removed from the rest of the safety catheter 10 it cannot be reattached to the handle 12.

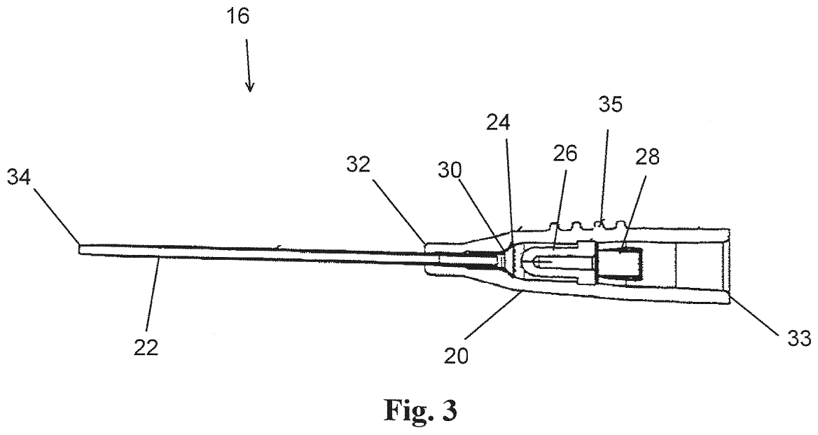

FIG. 2 illustrates an exploded view of the safety catheter 10 showing the various components of one version of the safety catheter 10. In addition to the shield 14, the safety catheter 10 includes a luer or hollow body portion 20, a cannula 22, an eyelet 24, and a one-way valve 26. In combination, these components comprise the luer assembly 16, which is shown and described in more detail with reference to FIG. 3. The safety catheter 10 further comprises a shuttle body assembly 50 including a stylet 36, a filter plug 40, and a shuttle body 38, which is shown in more detail in FIG. 4. FIG. 2 further illustrates an exploded view of a handle assembly 63 comprising a body top 54 engaged with a handle body 52 configured to retain a spring 70 therein. The handle assembly 63 further comprises an actuator 80 that is retained on the body top 54 and is configured for longitudinal movement relative thereto. In the illustrated version, the luer assembly 16, the shuttle assembly 50, and the handle assembly 63 interact with one another in multiple stages to provide a method of accurately and effectively accessing the vasculature of a patient and reducing the risk of accidental needle sticks after the vasculature has been properly accessed.

FIG. 3 illustrates a more detailed cross-section view of the luer assembly 16. In one version, the cannula 22 comprises a proximal end 30 and a distal end 34, where the proximal end 30 of the cannula 22 is bonded or otherwise attached to the eyelet 24. During assembly of the luer assembly 16, the eyelet 24, attached to the cannula 22, is fitted into the proximal end 33 of the hollow body portion 20 such that the cannula 22 extends from the distal end 32 of the body portion 20, as illustrated. After engaging the cannula 22 and eyelet 24 with the body portion 20, a one-way valve 26 may be positioned inside the hollow body portion 20 proximate the eyelet 24. The one-way valve, or other suitable blocking and/or selectively accessible component, allows for the stylet 36 of the shuttle assembly 50 (FIG. 4) to pass therethrough during operation of the safety catheter 10, but seals upon removal of the stylet 36 to prevent fluid from passing out of the luer assembly 16 until a proper attachment with a syringe, or the like, is created. In this manner, the luer assembly 16 can remain within the vasculature while various components are connected thereto via the one-way value for fluid delivery or removal.

The cannula 22 may be configured from any material, such as a flexible, bio-compatible elastomeric material, suitable for insertion into the vasculature of a patient. It will be appreciated that the cannula 22 may be transparent or semi-transparent to allow visualization of blood or other fluid, have any suitable internal diameter, have a bias toward a particular shape or configuration, be rigid or semi-rigid, and/or have any suitable geometry at the distal end 33 thereof. In an alternate version, the cannula 22 is integral with the eyelet 24 and/or one-way valve 26. It will be appreciated that the coupling of the components of the luer assembly 16 may be accomplished with any suitable engagement means such as with an adhesive, snap fit, friction fit, or the like.

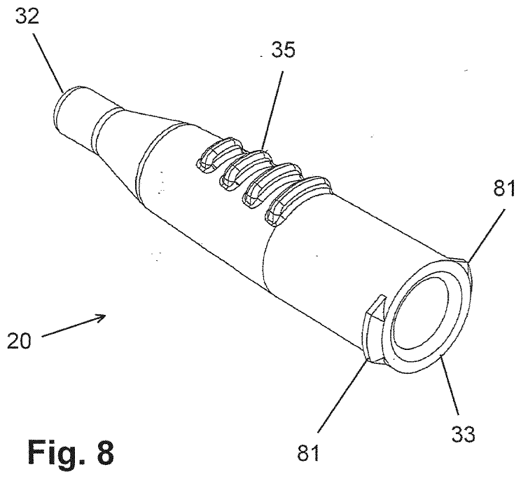

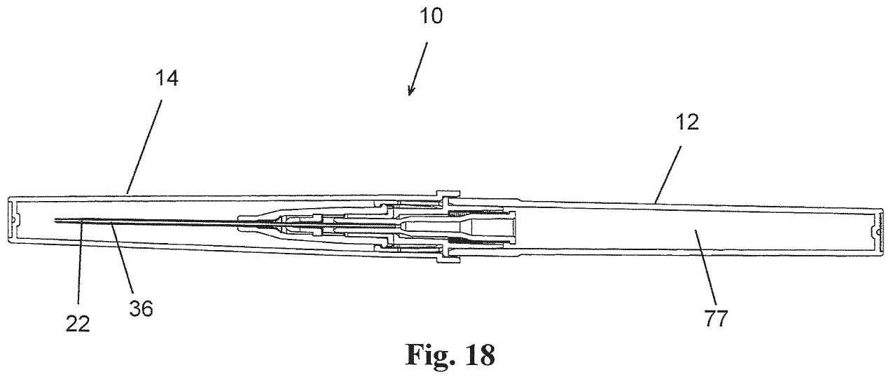

Still referring to FIG. 3, the illustrated version of the hollow body portion 20 has a generally frustoconical shape tapering from the proximal end 33 to the distal end 32. The proximal end of the eyelet 24 is configured to initially accept the stylet 36 and the distal end 46 of the shuttle body 38 during engagement of the luer assembly 16 and shuttle assembly 50. The outer surface of the luer or hollow body portion 20 may include a textured surface 35, such as a knurled surface or ridged surface, configured to be gripped by a user during operation of the safety catheter 10. The illustrated version of the hollow body portion 20 further comprises a pair of guides 28 configured to engage resilient fingers 66 on the body portion 54. It will be appreciated that the hollow body portion 20 may have any suitable shape or configuration designed to retain a cannula 22, to be advanced distally by a user, and/or to engage resilient fingers 66 associated with the body portion 54. It will be appreciated that the hollow body portion 20 may include any suitable number of guides 28, such as one or a plurality of guides, configured to engage a corresponding one or a plurality of resilient fingers 66. With reference to FIG. 1 and FIG. 8, the hollow body portion 20 further comprises a pair of lateral flanges 81 configured to engage the actuator 80 (FIG. 1), as will be described with more detail in reference to FIGS. 18-27a.

The luer assembly 16 is configured for removal from the safety catheter 10 and is designed to establish the site for fluid access into the vasculature of the patient. After the luer assembly has been properly positioned within a patient's vasculature, the other components of the safety catheter 10 will be removed such that an I.V. line, or the like, may be coupled with the luer assembly 16. It will be appreciated that the luer assembly 16 can include any suitable access means to the vasculature of a patient and/or means for coupling to a fluid delivery or extraction means.

FIG. 4 illustrates a cross-section view of one version of the shuttle assembly 50 associated with safety catheter 10. The shuttle assembly 50 comprises a shuttle body 38 having a proximal end 45 and a distal end 46. In the illustrated version, the distal end 46 of the shuttle body 38 comprises a channel 47 configured to retain a needle or stylet 36 having a proximal end 44 and a distal end 42. The channel 47 extends proximally from the distal end 46 of the shuttle body 38 along a portion of the shuttle body 38 and may be configured to retain the stylet 36 in any suitable manner such as, for example, with an adhesive, a bonding, a friction fit, or any other suitable mechanical engagement. In one version, the stylet 36 is integral with the shuttle body 38. The proximal end 45 of the shuttle body 38 comprises a laterally projecting annular flange 72 where, as shown in more detail in FIGS. 18-27a, the spring 70 is retained between the annular flange 72 and an annular flange 61 on the body top 54.

In one version, the shuttle body 38 is configured from a transparent or semi-transparent material to facilitate the visualization of fluid, such as blood, therein. In one version, the shuttle body 38 further comprises a cavity 49 defined by the outer wall of the shuttle body 38, the proximal end of the channel 47, and the filter plug 40. The stylet 36, in one version, has a lumen therethrough to facilitate the flow of blood, or other fluid, from the distal tip 42 to the proximal end 46 and into the cavity 49. Fluid entering the cavity 49 is trapped by the filter plug 40. In one version, at least the portion of the shuttle body 38 defining the cavity 49 is transparent, where upon accessing a patient's vasculature, blood will pass through the stylet 36 lumen and into the cavity 49 such that a clinician can see that the vasculature was successfully accessed. The stylet 36 may have any suitable configuration, such as a beveled distal tip 42, to facilitate access to a patient's vasculature. The filter plug 40 is configured to prevent fluid from passing out of the shuttle body 38 and may be integral with the shuttle body or bonded to the shuttle body 38.

Still referring to FIG. 4, in the illustrated version, the shuttle body 38 comprises a pair of opposing indents or flats 48 at or near the distal end 46. The flats 48 are configured to engage a corresponding pair of projections 68 located on resilient arms 66 associated with the body portion 54 (FIG. 5). It will be appreciated that the shuttle body 38 may include any suitable number of flats 48, or other coupling feature, configured to engage any suitable number of resilient arms 66 and/or projections 68. It will be appreciated that the features of the illustrated components are provided by way of example only, where any components suitable for facilitating the operation of the device in accordance versions and methods described herein are contemplated.

During assembly of the luer assembly 16 (FIG. 3) and the shuttle assembly (FIG. 4), the distal tip 42 of the stylet 36 is inserted into the proximal end 33 of the hollow body portion 20, through the one way valve 26, and through the flexible cannula 22. In this manner, the stylet 36 operates to stiffen the cannula 22 such that it can be inserted into the vasculature of the patient. The outer diameter of the stylet 36 may configured to be approximately the same as the inner diameter of the cannula 22 such that a seal is created between the cannula 22 and stylet 36, however, any suitable relationship between the cannula 22 and stylet 36 is contemplated. The stylet 36 may be inserted through the cannula 22 until the distal end 46 of the shuttle body abuts or nears the proximal end of the one way valve 26. The safety catheter 10 may include a cannula 22 and stylet 36 of any suitable length. In one version, the cannula 22 has a length that is approximately a centimeter shorter than the length of the exposed stylet 36 when initially engaged with the hollow body portion 20, however, any suitable dimension and relationship is contemplated.

Generally, the shuttle assembly 50 is configured to provide sufficient rigidity to the cannula 22 of the luer assembly 16 until the luer assembly 16 is properly positioned. Once positioned, as shown in more detail with reference to FIGS. 18-27a, the luer assembly 16 is removed from the shuttle assembly by initially advancing the luer assembly 16 with an actuator 80 and then manually removing the luer assembly 16 completely from the rest of the safety catheter 10. Once the shuttle assembly 50 is removed, the cannula 22 may regain its flexibility and the lumen of the cannula 22 will be clear for the transfer of fluid therethrough.

Referring to FIG. 5, one version of a handle assembly 63 is illustrated comprising a handle body 52, a body top 54, an actuator 80 (FIG. 17), and a spring 70. In the illustrated version, the body top 54 has a generally cylindrical proximal end 58 that is configured to be inserted into and bonded with the distal end 59 of the handle body 52. The body top 54 comprises an annular flange 61 having an outer portion 65 that abuts the distal end 59 of the handle body when engaged. The body top 54 and handle body 52 may have any suitable coupling including a bonding, a snap fit, a friction fit or, in an alternative embodiment, can be configured as an integral structure. The annular flange 61 of the body top 54 further comprises an inner portion 67 configured to retain a spring 70 within the handle body 52 in combination with the shuttle body 38 of the shuttle assembly 50 (shown in FIGS. 18-27a). More specifically, when the safety catheter 10 is assembled, the spring 70 is positioned between the annular flange 72 on the shuttle body 38 (FIG. 4) and the inner portion 67 of the annular flange 61. In one version, the spring 70 is used to selectively provide a motive force that is configured to translate the shuttle assembly 50 relative to the handle assembly 63 during operation.

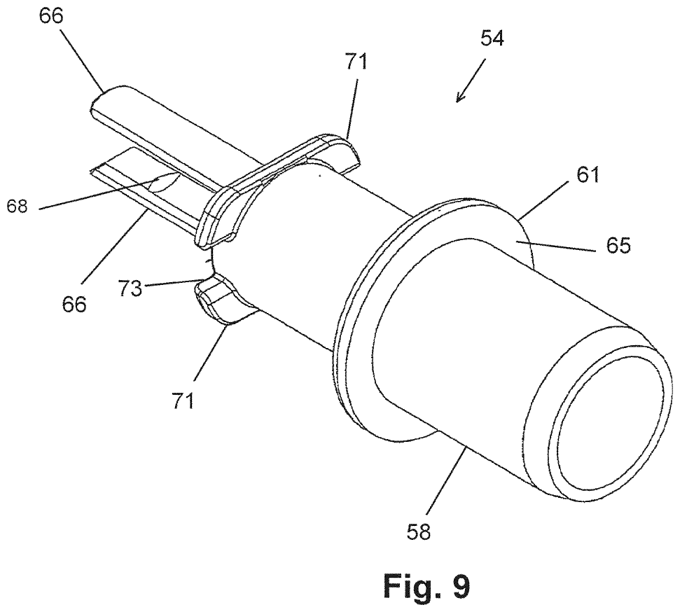

Referring to FIG. 9, a distal end 69 of the body top 54 comprises a pair of stops 71 projecting laterally outward from the body top 54. The stops 71 define a pair of gaps 73 (FIG. 2) therebetween. An actuator 80 is configured to engage the distal end 69 of the body top 54 and is configured to translate axially relative thereto. The operation of the actuator 80 relative to the body top 54 will be described in more detail with reference to FIGS. 17-27a. Projecting proximally from the distal end 69 of the body top 54 are a pair of resilient arms 66 having projections 68 projecting laterally inward from the distal ends thereof. The resilient arms 66, in the illustrated version, are configured to pivot as a living hinge about the connection point between the resilient arms 66 and the distal end 69 of the body top 54. The projections 68 are configured to engage the flats 48 on the shuttle body 38 of the shuttle assembly 50 as will be described in more detail with reference to FIGS. 18-27a.

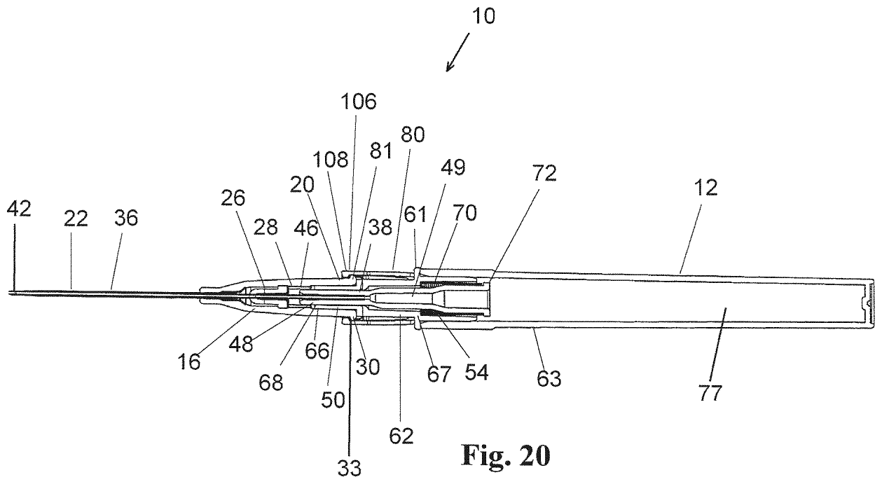

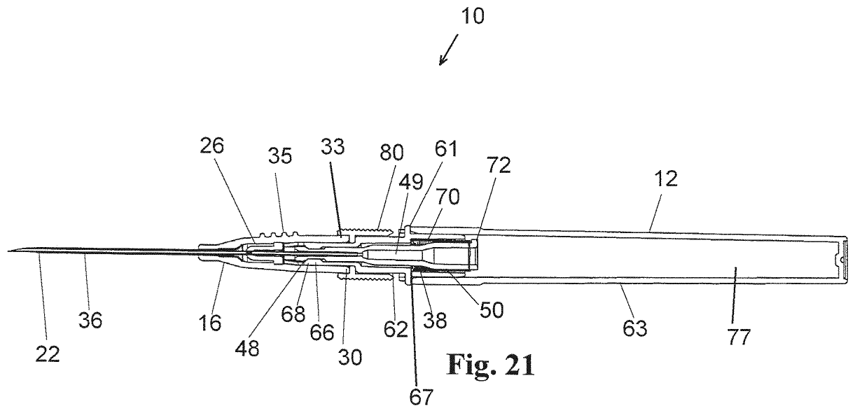

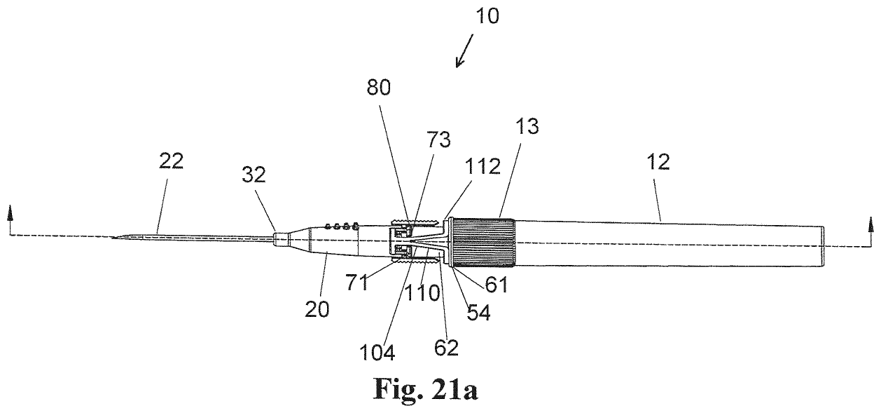

Still referring to FIG. 5, one, version of the body top 54 comprises providing a least a portion of the body top 54 that is transparent or semi-transparent. In one version, when the handle assembly 63 is engaged with the shuttle assembly 50, as will be described in more detail herein, the cavity 49 of the shuttle body 38 is aligned with the distal portion of the body top 54. With reference to FIGS. 20-21a, by providing a transparent distal portion 69 of the body top 54, which aligns with the transparent portion of the shuttle body 38 covering the cavity 49, a flash window 62 is created that allows a clinician to see that a patient's vasculature has been properly accessed. Providing a flash window 62 may eliminate a clinician having to guess as to the proper placement of the safety catheter 10 within the patient. After access to the vasculature has been confirmed, the safety catheter 10 may be further operated in accordance with FIGS. 18-27a. It will be appreciated that the luer assembly 16, the shuttle assembly 50, and the handle assembly 63 are described by way of example only, where any suitable components in any suitable configuration may be provided in accordance with versions described herein. Components may be separate or integral.

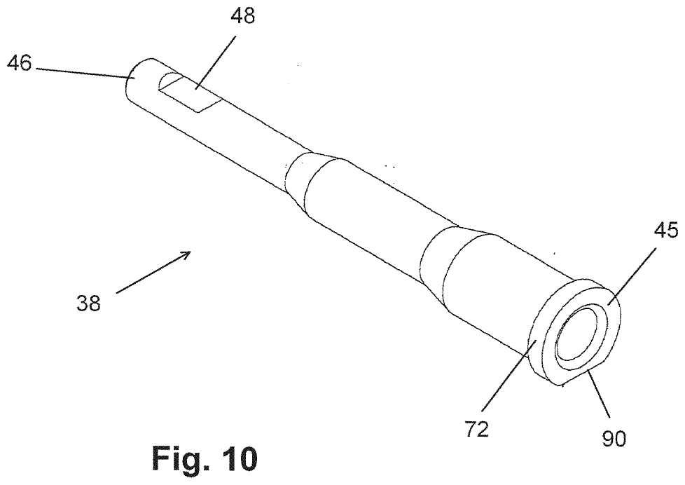

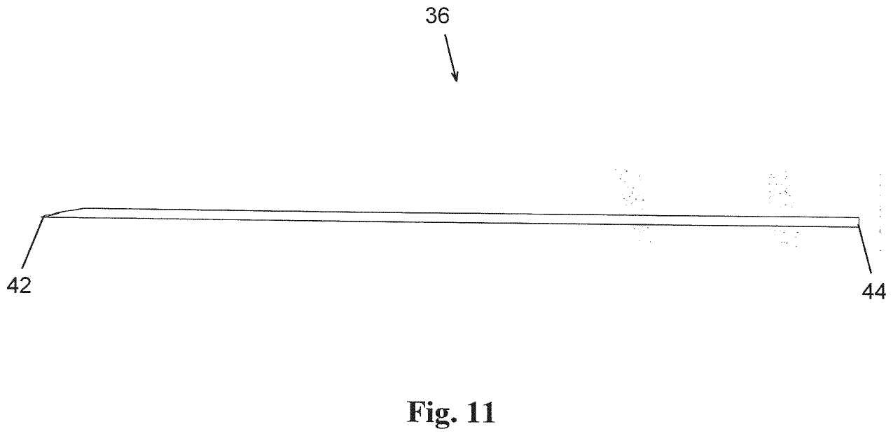

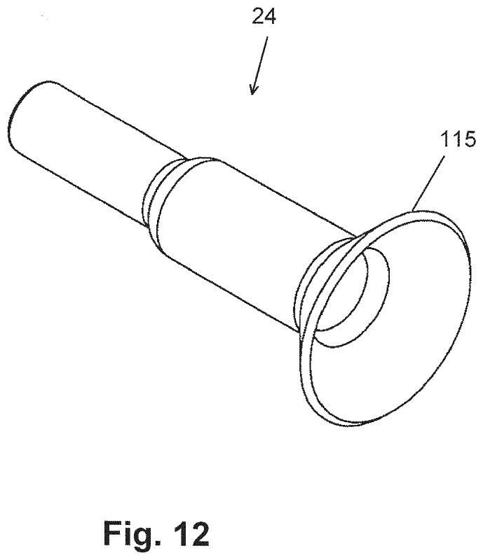

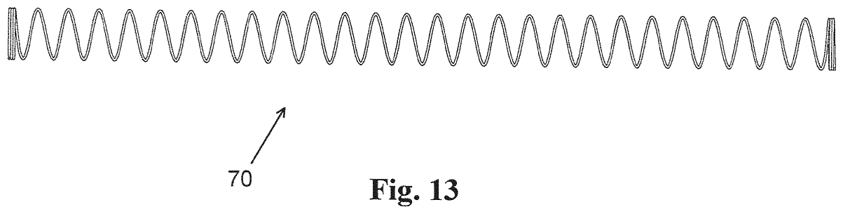

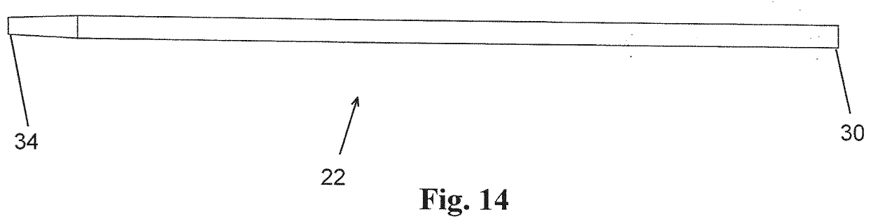

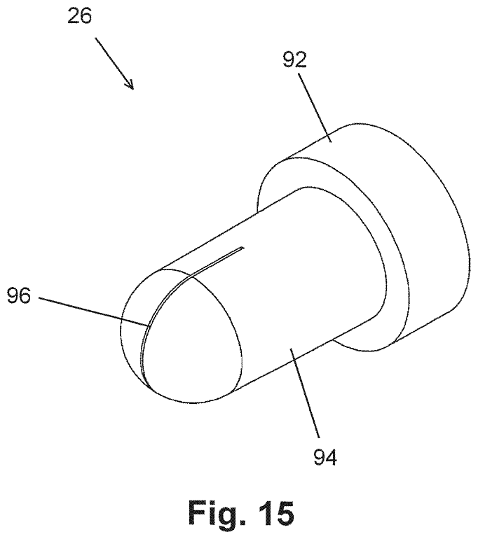

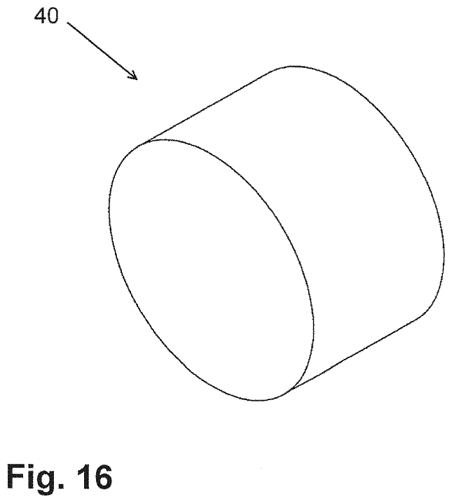

FIG. 6 illustrates a more detailed perspective view of the shield 14 and FIG. 7 illustrates a more detailed perspective view of the handle 12. FIG. 8 illustrates a more detailed perspective view of the hollow body portion 20. FIG. 9 illustrates a more detailed perspective view of the body top 54. FIG. 10 illustrates a more detailed perspective view of the shuttle body 38, where in one version the shuttle body 38 comprises a flat 90. FIG. 11 illustrates a more detailed side view of the stylet 36. FIG. 12 illustrates a more detailed perspective view of the eyelet 24 having, in one version a conical bevel 115. FIG. 13 illustrates a more detailed side view of the spring 70. FIG. 14 illustrates a side view of cannula 22, where in one version the cannula 22 comprises a distal end 34 having a taper. FIG. 15 illustrates a more detailed perspective view of the one-way valve 26. The one-way valve may be any suitable valve and may include, for example, an annular collar 92 and a distal valve portion 94 having a slit 96 therein. The valve portion 94 may be configured from any suitable material such that the slit 96 is normally sealed unless penetrated, for example, by the stylet 36 or other vasculature access or delivery device or component. It will be appreciated that any suitable valve or component that selectively restricts the movement of fluid is contemplated. FIG. 16 illustrates a more detailed perspective view of the filter plug 40. It will be appreciated that filter plug 40 may be configured from any suitable material and may have any suitable configuration to prevent or obstruct the flow of fluid while allowing displacement of air or another gas.

FIG. 17 illustrates one version of an actuator 80 having a proximal end 98 and a distal end 100. Actuator 80 comprises a base 95 having a dorsal thumb pad 97, a pair of distally extending rails 102, and a pair of lateral arms 104. The lateral arms 104 further comprise a pair of distal retention latches 106 having inwardly projecting lateral projections 108 and a pair of proximal living hinges 110. The proximal ends of the living hinges 110 are joined by a pair of crescent-shaped bands 112 that form a partial annular band at the proximal end of the actuator 80. The operation of actuator 80 will be described in more detail with reference to FIGS. 18-27a.

With reference to FIGS. 18-27a, one version of the operation of the safety catheter 10 is illustrated. Generally, the operation of the safety catheter is to transition the shuttle assembly 50 from a first position distal to the handle 12 to a second location inside the chamber 77 of the handle 12. More specifically, in one version, when the shuttle assembly 50 in its first location on the handle 12, the safety catheter 10 can be used to establish fluid access for the luer assembly 16 into the vasculature of the patient. To maintain this fluid access site, the luer assembly 16 is separated from the rest of the safety catheter 10. After separating the luer assembly 16 from the rest of the safety catheter 10, the shuttle assembly 50 is retracted to its second location inside the handle 12. When in the second position, the sharp distal tip 42 of the stylet 36 is effectively concealed inside the chamber 77 of the handle 12 to prevent inadvertent or accidental “sticks” by the stylet 36.

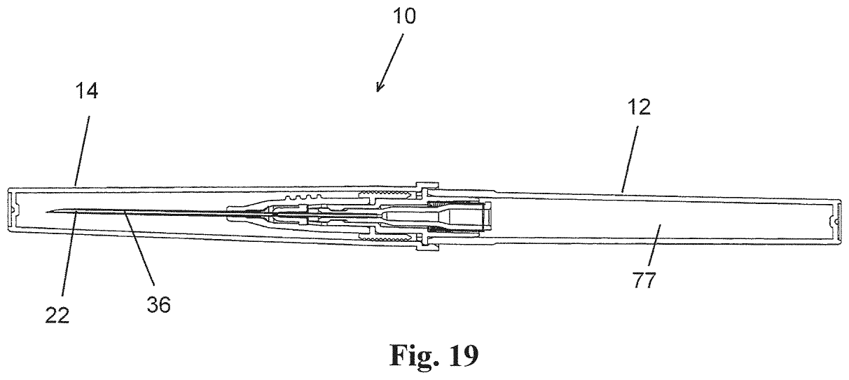

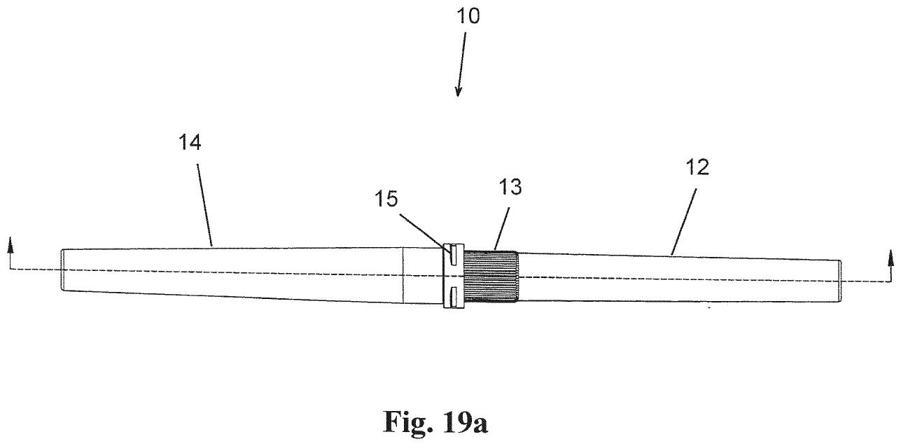

Referring FIGS. 18-19A, the safety catheter 10 is shown in it pre-use configuration with the shield 14 engaged with the handle 12 to effectively conceal the stylet 36. The safety catheter 10 may be packaged in any suitable manner for the safe transport and/or storage on the device.

FIGS. 20-21A illustrate one version of the safety catheter 10 after removal of the shield 14 therefrom such that the safety catheter 10 is in a configuration designed to access the vasculature of a patient. When the shuttle assembly 50 is in its first location, the luer assembly 16, the shuttle assembly 50 and the handle assembly 63 all interact with each other. As illustrated, the stylet 36 of the shuttle assembly 50 is retained within the cannula 22 of the luer assembly 16 and the distal end 46 of the shuttle body 38 is positioned proximal to and adjacent the one-way valve 26 of the luer assembly 16 within the guides 28. The stylet 36 extends distally from the shuttle body 38, through the one-way valve 26, and through the cannula 22. In the illustrated configuration, the stylet 36 stiffens the cannula 22 for insertion into the vasculature of a patient.

At the same time, the proximal end 33 of the hollow body portion 20 of the luer assembly 16 is positioned over the resilient fingers 66 of the body top 54, where the projections 68 on the resilient fingers are engaged, as best seen in FIG. 21, with the flats 48 of the shuttle body 38. As illustrated in FIGS. 20 and 21, positioning the hollow body portion over the resilient arms 66 maintains the projections 68 within the flats 48 such that the shuttle assembly 50 is unable to move relative to the handle assembly 63. This interaction between the luer assembly 16, the shuttle assembly 50, and the handle assembly 63 effectively holds the shuttle assembly 50 in its first location relative to the handle 12. While the shuttle assembly 50 is in its first location, as shown in FIGS. 20-21a, the spring 70 is compressed between the annular flange 72 on the shuttle body 38 and the annular flange 61 on body top 54. The spring 70 is configured to bias the shuttle assembly 50 proximally into the holder 12, however, the retention of the projections 68 of the resilient arms 66 within the flats 48 prevents the proximal retraction of the shuttle assembly 50. The spring 70 will remain compressed until the shuttle assembly is released from both the luer assembly 16 and the actuator 80.

Still referring to FIGS. 20-21A, the actuator 80 is shown engaged with the body top 54 and with the proximal end 33 of the hollow body portion 20 of the luer assembly 16. More specifically, in the illustrated version, the retention latches 106, having lateral projections 108, are engaged with the lateral flanges 81 on the hollow body portion 20. In this configuration, the luer assembly 16 is secured to the rest of the safety catheter 10. As best seen in FIG. 21a, the neck of the lateral arms 104 is positioned in the gaps 73 between the stops 71 on the body top 54. The bands 112 of the actuator substantially encircle the distal end of the body top 54 adjacent the annular band 61. In this position, the actuator 80 is secured to the body top 54 and the living hinges 110 of the lateral arms 104 are in a relaxed position, where only the necks of the lateral arms 104 are positioned within the gaps 73 between the stops 71 of the body top 54.

As shown in FIGS. 20-21A, the safety catheter is configured for insertion into the vasculature of a patient. Upon insertion of the cannula 22 and stylet 36 into the patient, versions herein comprise confirming that the safety catheter 10 has been properly positioned such that the luer assembly 16 is in fluid communication with the vasculature of the patient. After successfully accessing the vasculature, blood will pass through the lumen of the stylet 36 and into the cavity 49 within the shuttle body 38. Because, in one version, the shuttle body 38 and surrounding body top 54 are transparent, the blood will be visible through this flash window. Visualizing blood through the flash window 62 will indicate to the clinician that the vasculature has been properly accessed. The filter plug 40 confines the blood that enters into the cavity 49 of the shuttle assembly 50 and prevents blood borne pathogens from leaking out of the safety catheter 10.

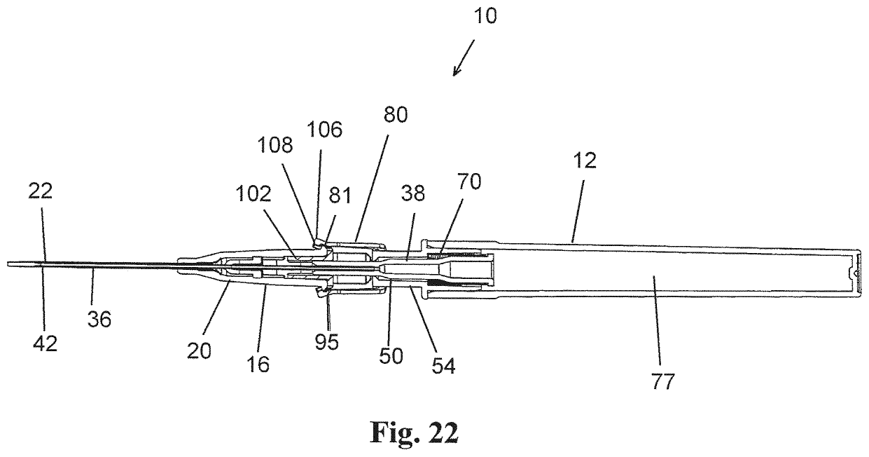

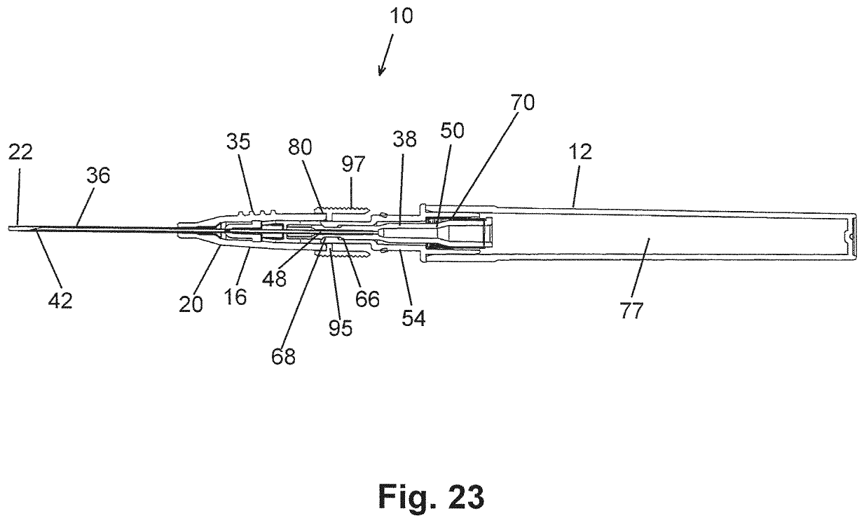

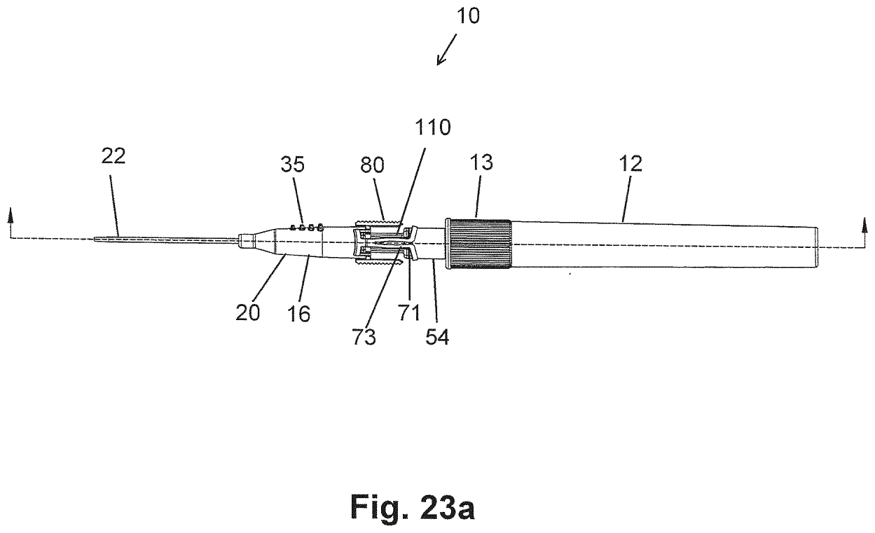

With reference to FIGS. 22-23A, after the vasculature of a patient has been accessed, the cannula 22 can be advanced beyond the distal tip 42 of the stylet 36 and/or farther into the vasculature. Advancing the cannula 22 is accomplished by the clinician placing, for example, their index finger on the dorsal pad 97 (FIGS. 23-23a) and distally advancing the actuator. As the actuator 80 is advanced, the retention latches 106 flex outward to disengage the lateral projections 108 from the lateral flanges 81 on the hollow body portion 20 of the luer assembly 16. This disengagement frees the luer assembly 16 for removal from the rest of the safety catheter 10. Concurrently, as the actuator 80 is advanced, the rails 102 push the luer assembly 16 distally, thus advancing the cannula 22 farther into the vascular of the patient. As the luer assembly 16 is pushed distally by the actuator 80, the base 95 of the actuator moves to cover the resilient arms 66 (FIG. 23) of the body top 54 such that the projections 68 are still retained within the flats 48 on the shuttle body 38. In this position, the hollow body portion 20 of the luer assembly 16 is no longer retaining the resilient arms, however, the actuator 80 prevents the resilient arms from expanding laterally to release and allow retraction of the shuttle assembly 50. In this manner the cannula 22 is extended further into the vasculature of a patient before allowing for the release of shuttle assembly. This configuration may be beneficial as it allows the cannula 22 to be advanced with some stiffness, and to be repositioned if necessary, before the stylet 36 is retracted into the handle 12. As the actuator 80 is advanced, the living hinges 110 (FIG. 23a) on the actuator are drawn and contracted through the gaps 73 between the stops 71 of the body top 54. This contraction biases the actuator 80 in a proximal direction, which upon release of the actuator, or by decreasing distal force on the actuator, will move the actuator proximally.

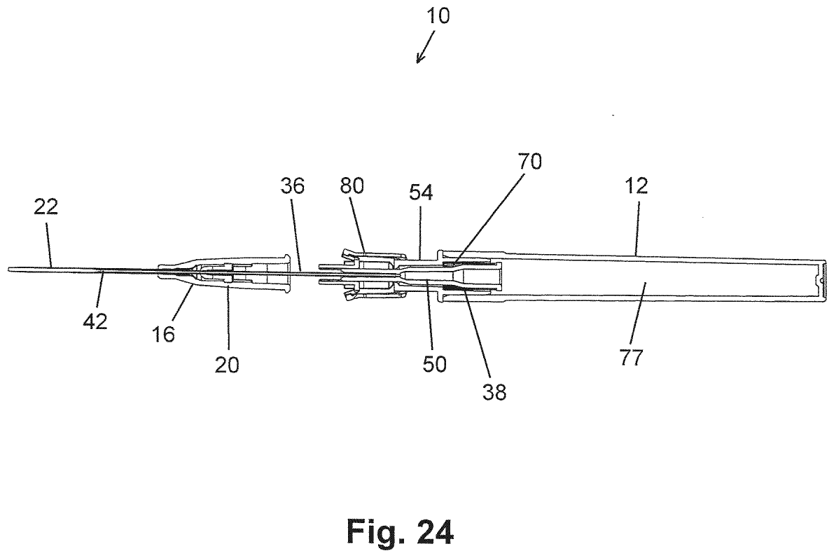

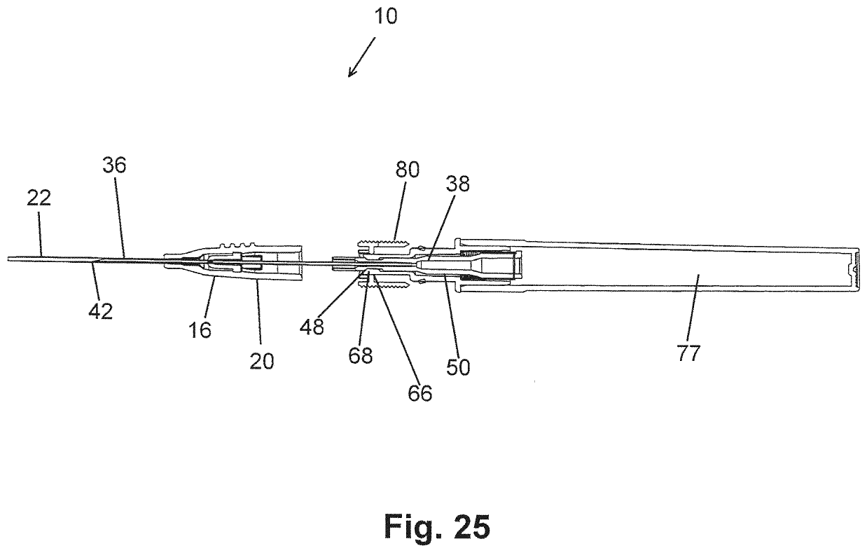

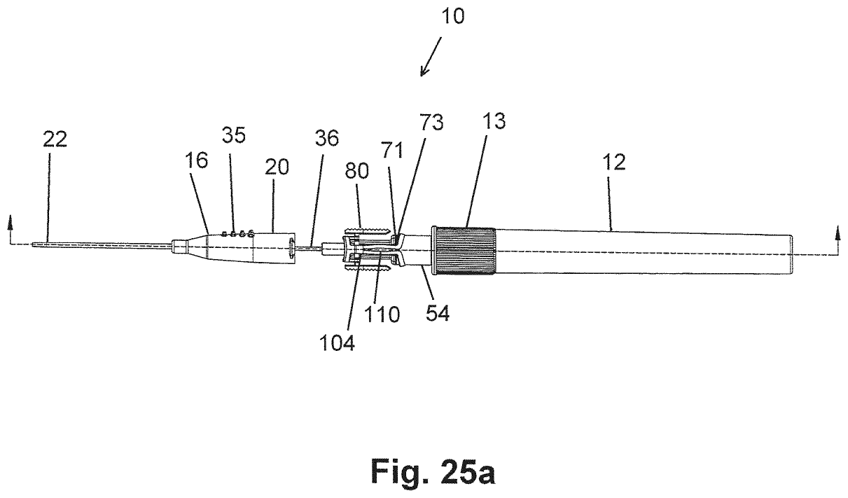

With reference to FIGS. 24-25A, after the cannula 22 has been further advanced, the luer assembly 16 can removed from the rest of the safety catheter 10 and remain within the vasculature of a patient. With the clinician's finger still positioned on the actuator 80, retaining the stylet 36 and shuttle assembly 50 in the first position, the luer assembly 16 may be guided off the stylet 36. As illustrated, the actuator 80 (FIG. 25) will maintain the projections 68 of the resilient arms 66 within the flats 48 until the actuator 80 is allowed to retract, thereby securing the shuttle assembly in the first position until release of the stylet is desired.

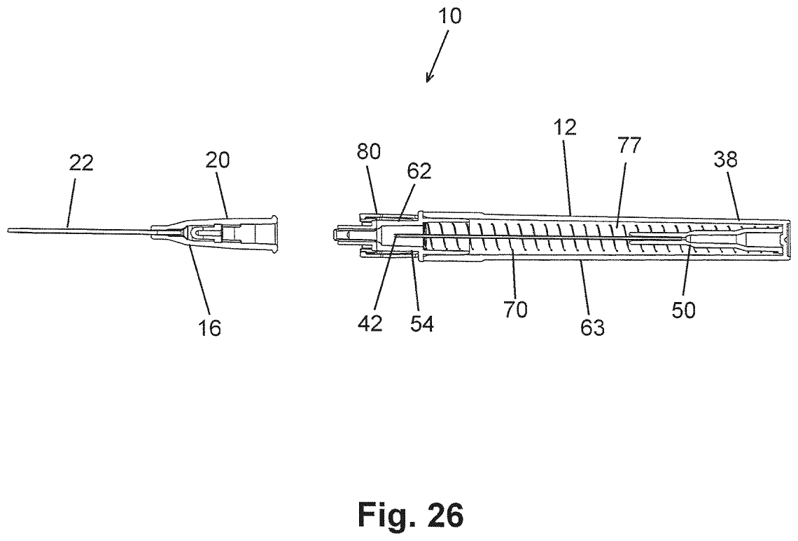

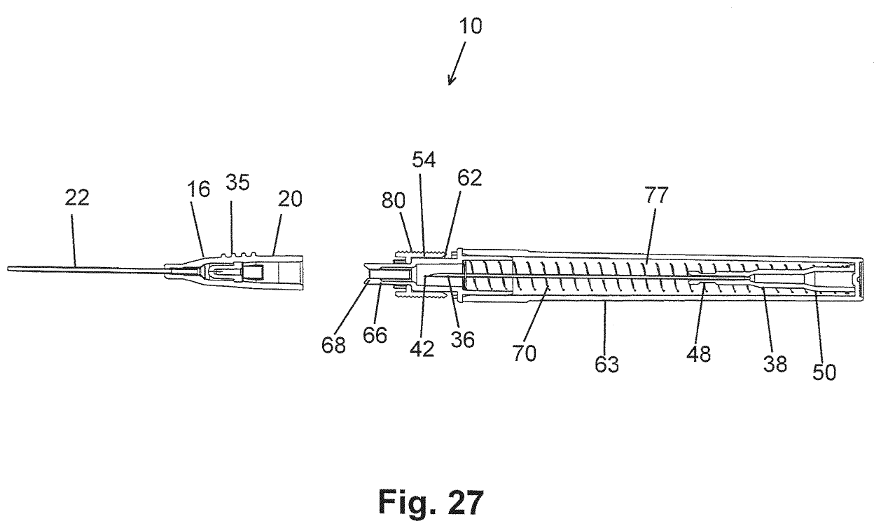

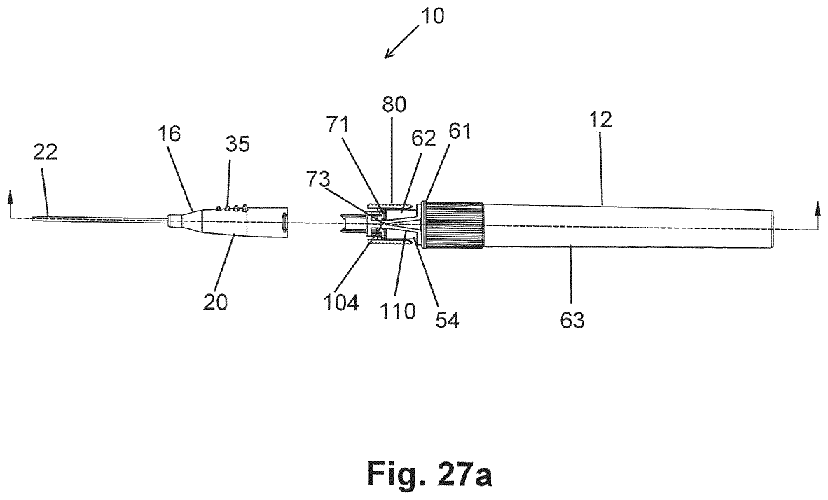

With reference to FIGS. 26-27A, the shuttle assembly 50 can be released for retraction into the handle 12 at any time after the luer assembly 16 has advanced. The luer assembly 16 can be partially or fully removed from the shuttle assembly 50 before allowing the shuttle assembly 50 to retract. Retraction is caused by the clinician releasing distal pressure on the actuator 80 such that the proximal bias of the living hinges 110 (FIG. 27a) urges the actuator proximally. As the actuator 80 moves proximally, the resilient arms 66 (FIG. 27), which are biased outwardly, are no longer retained within the flats 48. Once the resilient arms 66 are able to expand laterally, the projections 68 on the resilient arms disengage the flats 48. The shuttle body 38 of the shuttle assembly 50, once disengaged from the holder assembly 63, is urged to move proximally by the spring 70 retained within the handle 12. The spring 70 will urge the shuttle assembly 50 proximally into the chamber 77 of the handle 12, thus concealing the distal tip 42 of the stylet inside the handle 12. Concealing the stylet 36 in this manner can reduce the risks associated with accidental needle sticks. Once the shuttle assembly 50 is retained within the handle 12, in one version, the distal tip 42 of the stylet will be aligned with the flash window 62 on the body top 54. In this version, the clinician will be able to see the distal tip 42 and know that the stylet 36 is properly retained and no longer presents a risk.

FIGS. 28-38 present various side, partially cross sectional, schematic representations of devices for collecting blood and simultaneously inserting a catheter into a patient. Those of ordinary skill in the art will recognize that various aspects and features of FIGS. 28-35 bear similarities to features shown in FIGS. 1-27, and the embodiments of FIGS. 28-35 contemplate incorporation of such similar figures.

It is noted that the numbering system of FIGS. 28-38 is different than the numbering system of FIGS. 1-27. The use of a “2”, “3” or “4” prefix for an element number (NXX.X) refers to an element that is the same as the non-prefixed element (XX.X), except as shown and described. As an example, an element 320.1 would be the same as element 220.1, except for those different features of element 320.1 shown and described. Further, common elements and common features of related elements may be drawn in the same manner in different figures, and/or use the same symbology in different figures. As such, it is not necessary to describe the features of 220.1 and 320.1 that are the same, since these common features are apparent to a person of ordinary skill in the related field of technology. Further, it is understood that the features 220 and 420 may be backward compatible, such that a feature (4XX.X) may include features compatible with other various embodiments (2XX.X), as well as the inventions shown in FIGS. 1-27, as would be understood by those of ordinary skill in the art.

Referring to FIG. 28, a side elevational semi-cross sectional view of apparatus 220 is shown. In one embodiment, device 220 includes a catheter 230, a needle assembly 240, and a housing assembly 260. Preferably, these 3 components are aligned linearly along a common axis, although such linear arrangement is not required. Apparatus 220 in FIG. 28 is shown in position 220.1, which is the fully extended position. It can be seen that a sharp tipped cannula extends from a distalmost end. Cannula 242 is in fluid communication with an internal flexible tube 262 that extends to the proximal end of device 220. As shown in FIG. 28, the protective cap 14 has been removed from the distal end, such that the sharp tip is exposed. Device 220 as shown in FIG. 28 is ready to be inserted into the circulatory system of a biological unit by a user.

Referring to FIGS. 28-31, device 220 includes needle assembly 240 that is held in an extended position by the combined action of an adapter 261 and the body of catheter 230. Catheter body 230 includes an inner surface 233 that slides over the outer surface 261.4 of adapter 261, and slightly bends a pair of arms 261.2 toward an outer diameter of body 241. In a manner similar to that previously described, the arms 261.2 each include a projection 261.1 that is received within notches 241.1 of body 240. The projections are received snuggly within the notches, providing means for limiting retraction of needle assembly 4 while catheter 230 is kept on the distal end of device 220.

In the fully extended, prior to use configuration 220.1, the notches and projections physically interfere with one another to prevent movement of needle assembly 240. Although this restraint can be accomplished solely with interference, still further assistance in maintaining this interference is provided by the snug fit of the inner diameter of catheter assembly 230 over the outer diameter of the arms of the adapter. However, it is understood that various other embodiments contemplate limiting the relative movement of needle assembly 240 by friction only (such as friction against the inner diameter of the adapter), which in yet other embodiments is assisted by additional friction (and compression) by the inner diameter of the catheter body around the arms of the adapter. This retention is in a manner similar to that described previously with regards to the projections 68 of resilient fingers 66 being located within indents 48. As shown and discussed herein, it is understood that means for limiting retraction, as well as means for retaining in a position, and means for axially restraining, can be by interference between features of adjacent components, or friction between adjacent components, or combinations of both.

So long as catheter 230 is kept in place as shown in FIG. 28, the flexible arms 261.2 are maintained in an interlocking manner with needle assembly 240. However, once catheter 230 is removed, the arms 261.2 are free to slightly flex outward, and out of interlocking engagement and interference with notches 241.1. Still further, it can be seen that in some embodiments the projections 261.1 and notches 241.1 have beveled leading and trailing surfaces. These surfaces are beveled in such a manner that any axial movement of needle assembly body 240 relative to adapter 261 will result in a force that attempts to radially outwardly displace arms 261.2, even if the arms are not pre-biased to flex outward.

Although a means for limiting retraction of (or axially restraining) needle assembly 240 has been shown and described, still other means for limiting retraction are contemplated. For example, the arms 261.2 can be biased radially inward (i.e., toward the device centerline), neutrally biased (i.e., oriented parallel to the outer surface the needle body), or biased outwardly (i.e., biased away from engagement of the notches with the projections). Still further, although beveled leading and trailing edges have been shown and described, the present invention includes any type of shapes for the notches and projections, including hemispherical projections received within hemispherical dimples, square-edged leading and trailing edges fitting within squared-edged notches, or the like. Further, it is not necessary that the projections and notches be complementary in shape, such as, for example the use of squared-edged projections within curving, rounded, or hemispherical notches.

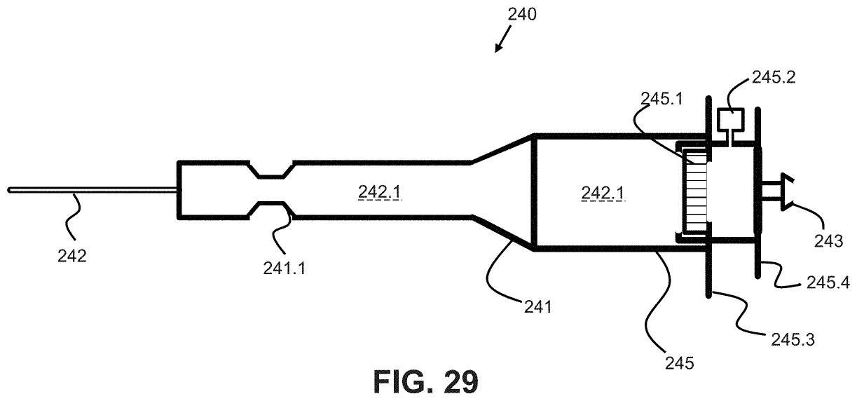

Referring to FIG. 29, a needle 240 is shown, which includes an internal flowpath 242.1 (partially shown), that extends from the distal tip of cannula 242 to a proximal connector 243. In a manner well known, the insertion of needle assembly 240 into a circulatory system provides a path for the circulated fluid through the cannula. Briefly referring to FIG. 28, this fluid path continues through a tube 262 all the way to a connector 263. Fluid received from cannula 242 passes within the needle body 241, and through a filter 245.1 located within a casing 245 at the proximal end of assembly 240. This optional filter 245.1 preferably keeps any particulates within flowpath 242.1 from reaching the fluid collection device. Still further, needle assembly 24 includes a one-way valve 245.2 that assists in purging air from flowpath 242.1. Device 245.1 is a porous filter, preferably of the type in which the porosity of the filter is maintained only so long as the filter does not contact flood, one example of such a material being Porex® material. Upon contact with blood, the filter loses porosity, such that there can be no further leakage of either trapped air or blood through device 245.2.

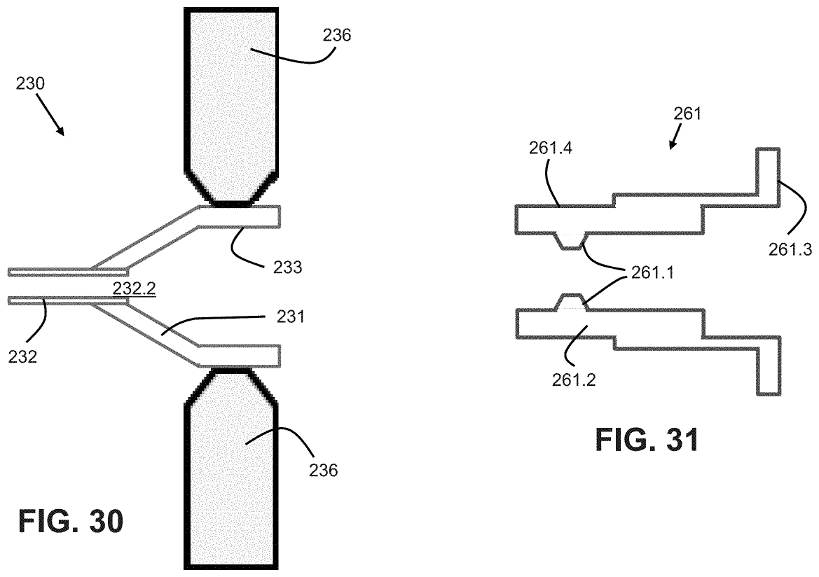

FIG. 30 shows a top view of a partially cross sectioned catheter assembly 230. Catheter assembly 230 includes a body 231 which includes a proximal portion that includes external butterfly wings 236 and a luer fitting 233, and a distal section including a flexible lumen 232 which is adapted and configured to provide fluid communication with the circulatory system of a biological unit.

Preferably, the butterfly wings 236 are attached to the outer diameter of body 231, and extend radially outward on opposite sides of assembly 230. These wings 236 function as handles by which a user's fingers can grip assembly 230. For example, after cannula 242 and the distal tip of lumen 232 have been inserted into the circulatory system, the user can pinch or fold the flexible wings together (such as with the thumb and fore finger of a single hand) and hold the catheter in position as the assembly of the needle assembly 240 and housing assembly 260 are moved out of engagement with the catheter body. This manipulation permits the user to statically maintain flexible lumen 232 in fluid communication with the circulatory system, and at the same time remove cannula 242 from the circulatory system.

FIG. 31 is a cross sectional representation of an adapter 261 according to one embodiment of the present invention. Adapter 261 provides an interface from protective casing 265 to the proximal ends of needle assembly 240 and catheter assembly 230. As previously discussed, a pair of flexible arms 261.2 (similar to resilient fingers 66 discussed earlier) extend axially forward from a circular flange at the proximal end. This flange includes a proximal face that is preferably attached to a distal face 265.4 of protective casing 265. It is understood that the arrangement of the flexible arms and the other features of adapter 261 can be incorporated into an apparatus such as apparatus 220 in a variety of manners. Two such examples include multiple separate components that are adhered together, cast together or separately, 3-D printed separately or together, or the like. It is further understood that the adapter 261 is similar to the body top 54 previously discussed, and various features of body top 54 can be incorporated into an adapter such as adapter 261. Still further, although it has been shown and described that the assembly of adapter 261, needle assembly 240, and catheter assembly 230 provide not only for means for limiting retraction, but also means for permitting axial movement, similar to the body top 54 and actuator 80 previously shown and described. Various embodiments of the present invention include features and aspects of actuator 80 in devices such as apparatus 220.

It is further understood that adapter 261 is preferably attached to, or an integral part of, protective casing 265. In the various embodiments described herein, protection of the user is provided by retracting the sharp tip of the cannula into the interior of any portion of the device that remains attached to the housing after the needle retracts to the interior, protected position. As one example, a placement of the needle between projections 261.1 would provide protection from the sharp tip to the user. Still further, the reference to the first distal compartment, the compartment that encloses the retracted needle, includes the interior space up to the distalmost front faces of the flexible arms 261.2.

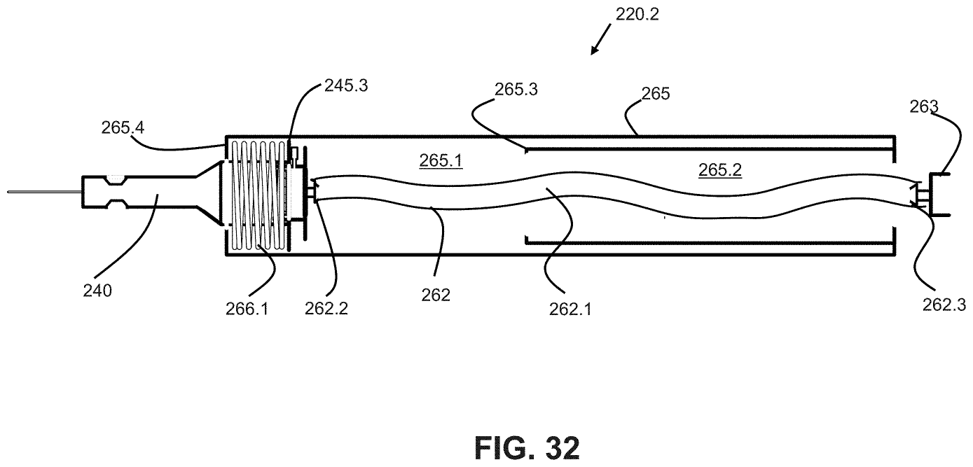

FIG. 32 is a side elevational view in partial cross section of portions of the apparatus shown in FIG. 28. In FIG. 32, catheter assembly 230 has been removed. This permits the arms 261.2 of adapter 261 (removed from FIG. 32 for clarity) to no longer provide means for limiting retraction or means for axial restraint of needle assembly 240, the force of compressed spring 266 being larger than any frictional or interfering force attempting to maintain the position of assembly 240. Therefore, needle assembly 240 in FIG. 32 is shown ready to move into its second, retracted position. This retraction is accomplished by a spring 266 shown in the compressed state 266.1. One end of this spring pushes against an inner, distal face 265.4 of casing 265, and the other end of the spring pushes against a surface of shoulder 245.3 of needle assembly 240 (as best seen in FIG. 29). Spring 266 in its compressed state biases needle assembly 240 to the retracted position.

FIG. 32 shows a casing 265 that includes a first compartment 265.1 that is preferably axially aligned with a second internal compartment 265.2. In one embodiment, the casing 265 is generally cylindrical, and a second, smaller internal cylinder establishes the length of the second compartment 265.2, such that the remainder of protective casing 265 is established as the first compartment 265.1. Although shown and described as an internal cylinder, various other embodiments contemplate any manner of delineating and separating the two compartments, including simple internal stops or abutments located within the inner diameter or interior of casing 265. This demarcation between the first and second compartments establishes the retraction limit of needle assembly 240 within casing 265.

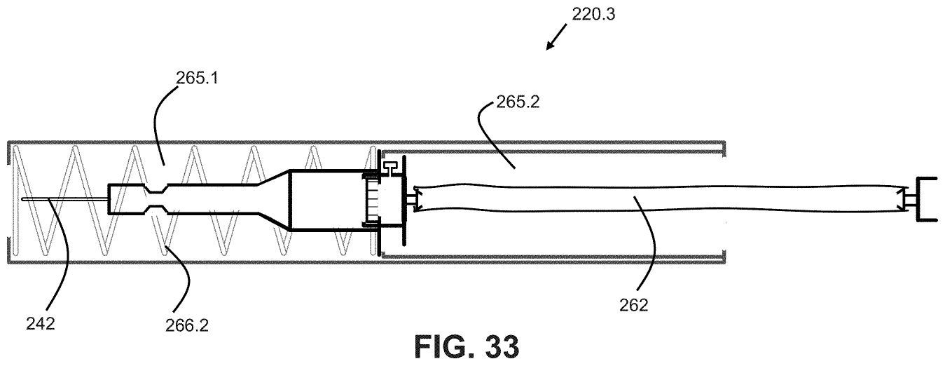

As will be seen in FIG. 33, a second compartment that is established by a thicker wall, or alternatively, a separate internal cylinder, open on both ends, and supported from the proximal end of casing 265, can provide, in combination with a cylindrical annular member 245.3, a physical separation between a first compartment that contains the used needle, and the open second compartment. This physical separation further minimizes any splashing or ejection of blood toward the opened proximal end of the second compartment (i.e., toward the user). In some embodiments, there can be a resilient seal (not shown) between the proximal face of annular member 245.3 and the travel stop or proximal end 265.3 to further minimize any splashing of blood.

Further, although various aspects of protective casing 260 have been shown and described as being cylindrical, it is understood that various embodiments of the present invention contemplate any outer or internal shapes, including as one example an external shape including ridges or dimples to act as finger or hand grips.

FIG. 32 further shows that the end cap 267 has been removed from the proximal end of casing 265, such that the internal flexible tube 262 can be extended outward and rearward from second compartment 265.2. As long as cap 267 remains in place, flexible tube 262 is placed in a slightly compressed, curving shape, as seen in FIG. 28. Preferably, the free length of tube 262 is greater than the combined length of the compartments 265.1 and 265.2. However, yet other embodiments contemplate a tube having a free length that is about the same as the distance traveled by the needle assembly from the fully extended position to the fully retracted position. Removal of end cap 267 permits relief of this compressive state, such that the collection vial fitting 263 extends outward for manipulation by the user. In some embodiments, Fitting 263 is a luer fitting, but can be of any type of fitting through which the connected vial receives fluid from the circulatory system of the biological unit through flowpath 262.1. During usage, cap 267 would be removed and the collection vial placed in fluid communication with path 262.1 while cannula 242 is in fluid communication with the circulatory system, and prior to removal of needle assembly 240 from the inserted catheter assembly 230. Various embodiments contemplate fitting 263 of any type that facilitate removal of fluid from the circulatory system.

FIG. 33 schematically shows the needle assembly 240 in its fully retracted position. Spring 266 has expanded to its fully released state 266.2, the final installed length of spring 266 being established by the abutment of travel stop 245.3 against travel stop 265.3. Preferably, the free length of spring 266 is longer than the length of first compartment 265.1, so as to maintain a net force holding the needle assembly in the retracted position. The sharp tip of cannula 242 is preferably located within the interior of casing 265, and fully within first compartment 265.1, although other embodiments are not so limited, and contemplate maintaining the distal end of the first needle in any protected space, such as within adapter 261 or any other components that remain attached to the device after needle retraction occurs. FIG. 33 shows the apparatus in the retracted state 220.3, after full retraction of the needle assembly, and after removal of the collection vial. The user can now dispose of assembly 220. If desired, the extended flexible tube 262 can be pushed back into cavity 265.2, and the cap 267 replaced on the proximal end to maintain the tube in the compartment for safe and convenient disposal.

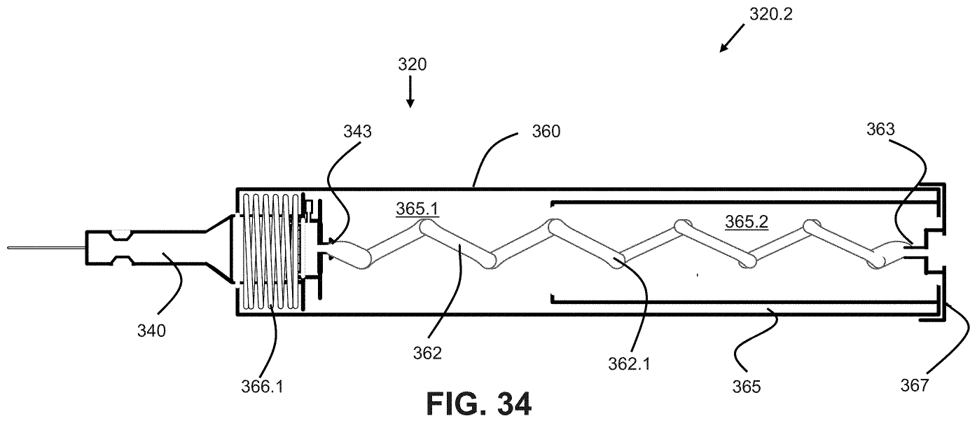

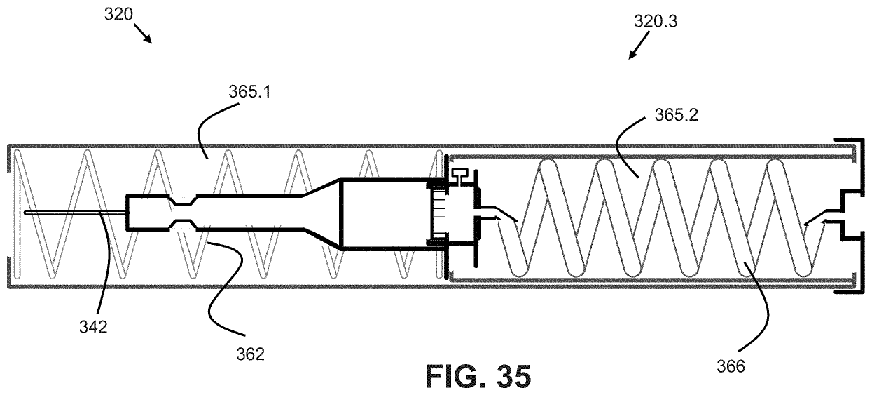

FIGS. 34 and 35 show views of an apparatus 320 according to another embodiment of the present invention. It is recognized that apparatus 320 uses a common numbering system with apparatus 220, with like features being identified by similar digits XX.X. Still other similar features can be identified visually within these figures. Some of the differences between device 320 and 220 will now be explained.

FIG. 34 shows device 320 in the extended position, prior to usage (similar to FIG. 28). Adaptor 361 is shown attached (or integral) with the proximal end of protective casing 365. The catheter assembly 30 is radially compressing and maintaining the arms 361.2 into notches 340.1, so as to maintain needle assembly 340 in the fully extended position. Needle assembly 340 is shown being biased to the retracted position by a coil spring 366 in a compressed state 366.1.

A flexible tube 362 is shown connecting the connector port 343 to the vial tubing port on connector 363. Apparatus 320 preferably includes a coiled tube 362 extending within portions of the first and second compartments 365.1 and 365.2. As shown in FIG. 34, tube 362 is fully extended and in a state of tension, with the spacing between coils being expanded, and the diameter of the coils being reduced. Extended tube 362 applies a force that biases toward each other the needle assembly 340 and the collection fitting 363. Because of this pulling force, collection port 363 is attached to the proximal end of casing 360 in order to maintain the collection port in position at the proximal end of casing 365. Fitting 363 now extends in some embodiments as a circular plate across the otherwise opened end of casing 365. The end cap 367 is now of reduced size in some embodiments, and covers and protects only the vial fitting (or luer fitting) itself, as well the tip of any needle 362.2 contained therein.

FIG. 35 shows needle assembly 340 in the fully retracted position. Similar to as seen before, spring 362 is likewise in its fully extended state, with its length limited by the travel stops. Tubing 362 has likewise changed length, and is now shown in its retracted state. The spacing between coils has decreased, and the outer diameter of the tube has increased. In some embodiments, a reduced state of tension is still maintained in tube 362 in this retracted state. Therefore, it can be seen that during the retraction of needle assembly 240, the needle assembly is both pushed by spring 362, but also pulled by tubing 366. Preferably, the free length of coiled tube 366 is less than the length of the second compartment 365.2. As shown in FIG. 35, the assembly 230 is ready for disposal.

It is further understood that a coiled flexible tube can be utilized in devices such as device 220. Referring to FIG. 28, such a coiled spring would be in a mild state of compression when the device is fully extended. The free length of this alternative coil spring in some embodiments would preferably be greater than the combined length of the first and second compartment. Referring to FIG. 32, if the end cap is removed the compressed coil spring would extend out of the proximal end of the device, thus eliminating any circumstances under which the user of the device would have to reach into the second compartment to pull out the tube. It is noted that the difference in length between tube 362 and the ready to use position 362.4 and the retracted position 362.7 is about the same as the net distance of retraction of the needle assembly.

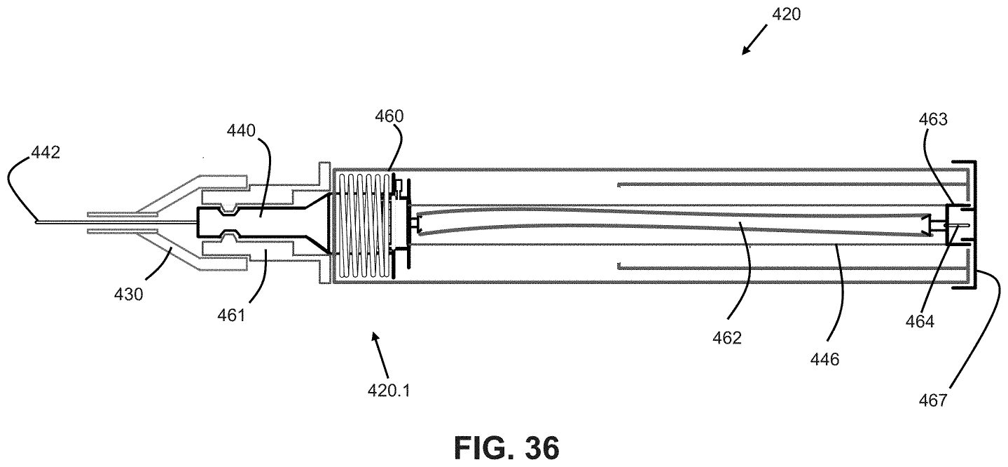

FIGS. 36-38 show views of an apparatus 420 according to another embodiment of the present invention. It is recognized that apparatus 420 uses a common numbering system with apparatus 220 or 320, with like features being identified by similar digits XX.X. Still other similar features can be identified visually within these figures. Some of the differences between devices 420, 320, and 220 will now be explained.

FIG. 36 shows an apparatus 420 in the fully extended, unused state. Apparatus 420 is similar to apparatus 220, except that flexible tube 462 is contained within a rigid protective sheath 446. Sheath 446 is coupled to the end of needle assembly 440, and moves from the extended to retracted positions concurrently with needle assembly 440. As shown in FIG. 36 in the fully extended position, sheath 446 is contained entirely within the first and second compartments of housing assembly 460. It is further understood that in yet other embodiments there is a single tube that provides both fluid communication between needles 442 and 464, and further a fixed connection between the body of the needle assembly and the fitting 463.

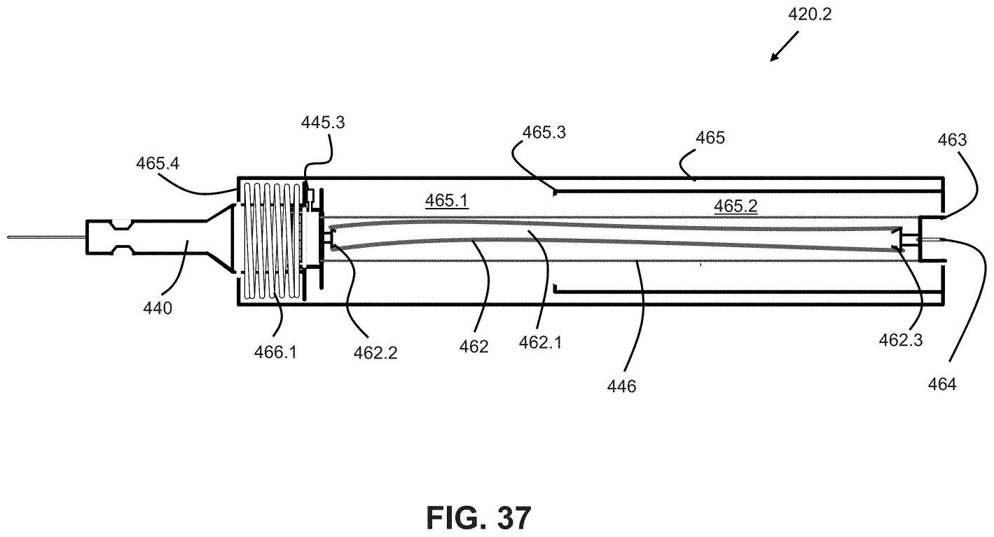

The proximal end of sheath 446 incorporates a fitting 463 adapted and configured for easy coupling and decoupling from the blood collection vial. In some embodiments, fitting 463 includes a second cannula 464 which pierces a seal on the inlet of the collection vial. This hollow second cannula 464 establishes fluid communication between the interior of the vial (which may be evacuated) and the hollow cannula 442. FIG. 37 shows the apparatus 420.2 in the extended state, with the end cap 467 removed. After removal of the end cap 467, the blood collection vial can be attached to fitting 463.

FIG. 37 shows device 420 in the extended position, but after usage (similar to FIG. 32). Further, adaptor 461 is not shown for sake of clarity. Needle assembly 440 is shown in the extended position, although being biased to the retracted position by a coil spring 466 in a compressed state 466.1.

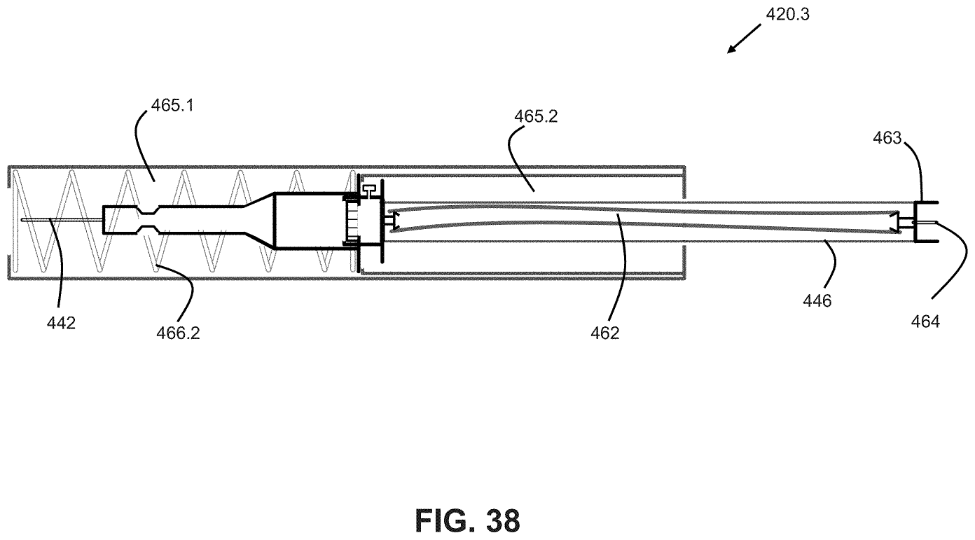

FIG. 38 shows device 420.3 in the fully retracted, post-usage state. As shown previously with apparatus 220.3, the first cannula 442 is contained entirely within the first compartment 465.1. Because of the connection of sheath 446 to needle assembly 440, this retractive movement results in sheath 446 extending rearward, with fitting 463 now extending out of compartment 465.2 at the proximal end of device 420.3. In some embodiments, cap 467 can be placed back onto fitting 463, so as to prevent inadvertent contact with second cannula 464.

Various aspects of different embodiments of the present invention are expressed in paragraphs X1, X2, and X3 as follows:

X1. One aspect of the present invention pertains to a device for collecting blood in a container and connecting to intravenous tubing. The device preferably includes a first distal compartment generally aligned with a second proximal compartment. The device preferably includes a needle assembly having proximal and distal ends and including a needle defining a first lumen and having a sharp tip on the distal end, the needle assembly being slidable within the first compartment. The device preferably includes a spring biasing the needle assembly toward a retracted position. The device preferably includes a tube in fluid communication with the first lumen and extending toward the second proximal compartment. The device preferably includes a sheath containing therein the tube, the sheath being slidable with the needle assembly; and a catheter assembly having a flexible second lumen supported by a body, the second lumen surrounding at least a portion of the needle.

X2. Another aspect of the present invention pertains to a device for collecting blood in a container and connecting to intravenous tubing. The device preferably includes a first compartment and a second compartment. The device preferably includes a needle assembly having proximal and distal ends and including a needle being slidable between an extended position in which the sharp tip is external to the housing and a retracted position. The device preferably includes means for biasing the needle assembly to either the retracted or extended position. The device preferably includes a fluid conduit in fluid communication with the first lumen and extending toward the proximal end of the device. And a catheter assembly having a flexible second lumen supported by a body, the second lumen surrounding at least a portion of the needle, the catheter assembly including a pair of opposing flexible wings adapted and configured for grasping by a user.

X3. Yet another aspect of the present invention pertains to a device for collecting blood in a container and connecting to intravenous tubing. The device preferably includes a first compartment and a second compartment. The device preferably includes a needle defining a first lumen and having a sharp tip on the distal end, the needle being slidable between two positions. The device preferably includes a flexible member biasing the needle assembly toward a position. The device preferably includes a tube in fluid communication with the first, the proximal end of the tube having a fitting adapted and configured for readily attaching and releasing the container for collecting blood; and a catheter assembly having a second flexible lumen supported by a body, the second lumen surrounding at least a portion of the needle.

Yet other embodiments pertain to any of the previous statements X1, X2, or X3, which are combined with one or more of the following other aspects. It is also understood that any of the aforementioned X paragraphs include listings of individual features that can be combined with individual features of other X paragraphs.

Which further comprises a gas purging valve that permits expulsion of trapped gas within the needle assembly.

Which further comprises a filter having an inlet in fluid communication with the first lumen and an outlet in fluid communication with the inlet of the tube.

Which further comprises means for retaining the needle assembly in the ready to use position.

Which further comprises a readily removable cap covering the proximal end of the device.

Wherein the spring is a coil spring that is either compressed when the first needle is in the extended position, or extended when the first needle is in the extended position.

Wherein the catheter assembly includes a fitting for readily attaching and releasing intravenous tubing, including luer-type fittings, threaded fittings, bayonet-type fittings, or the like.

Wherein the housing includes an abutment that limits the sliding of the needle assembly to the retracted position; preferably the abutment being a surface of a sliding component or a surface of a stationary component into which the sliding component slides.

Wherein the needle assembly abuts the distal end of the second compartment in the retracted position.

Wherein the catheter assembly includes a pair of opposing flexible wings adapted and configured for grasping by a user.

Which further comprises a rigid sheath containing therein the first tube, the rigid sheath preferably being slidable with the needle assembly from the extended position to the retraced position. Preferably the rigid sheath has a length that is about the same as the length of the tube contained therein, although other embodiments contemplate rigid sheaths that are longer or shorter than the tube contained therein.

Which further comprises a rigid sheath surrounding the tube, the rigid sheath and the tube being located within the housing when the needle assembly is in the extended position, and the proximal end of the tubing and the proximal end of the rigid sheath extending out of the second compartment when the needle assembly is in the retracted position.

Wherein the tube comprises a flexible material and has a free length that is less than the length of the second compartment, the tube being extended beyond the free length when the needle assembly is in the extended position.

Wherein the tube comprises a flexible material and has a free length that is greater than the length of the second compartment, the tube being compressed from the free length and fitting within the second compartment when the needle assembly is in the extended position.

Wherein the tube has a free length that is greater than the combined length of first compartment and the second compartment.

Wherein the tube has a first length prior to use of the device, a second length after the device has been used and the tube is free to extend at the distal end of the device, and the difference between the first length and the second length is about the same as the distance traveled by the sliding needle assembly when moving from the extended position to the retracted position.

Which further preferably comprises a fitting on the end of the tube within the second compartment, the fitting being adapted and configured for readily attaching and releasing the container for collecting blood. The fitting can be of any type suitable for blood collection vials, including screw-type, press to fit-type, bayonet-type, and others.

Wherein the proximal end of the tube has a fitting adapted and configured for readily attaching and releasing the container for collecting blood.

Wherein the fitting includes a second needle adapted and configured for puncturing a seal of the container for collecting blood. Preferably, the second needle extends a short distance from the fitting, and the tip of the needle is safely enclosed by the endcap when the device is in the ready to use configuration.

Wherein the needle is a first needle, and which further comprises a second needle in fluid communication with the tube and adapted and configured for receiving blood from the tube, the second needle being located on the proximal end of the device.

Wherein the first needle slides linearly along an axis from the extended position to the retracted position.

Wherein the sharp tip of the first needle extends out from the distal end of the second lumen when the first needle is in the extended position.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. A device for collecting blood in a container and connecting to intravenous tubing, comprising:
a housing having proximal and distal ends and a first distal compartment generally aligned with a second proximal compartment;
a needle assembly including a body supporting a needle defining a first lumen and having a sharp tip on the distal end, a portion of said needle assembly being slidable within the first compartment between an extended position in which the sharp tip is external to said housing and a retracted position in which the sharp tip is located within the first compartment;
a spring biasing the body of said needle assembly to the retracted position;
a tube in fluid communication with the first lumen and extending from the proximal end of said needle assembly through the first distal compartment and extending into the second proximal compartment;
a rigid sheath containing therein said first tube, said rigid sheath being slidable with said needle assembly from the extended position to the retracted position; and
a catheter assembly having a flexible second lumen supported by a second body, the second lumen surrounding at least a portion of the needle.

2. The device of claim 1 which further comprises a gas purging valve that permits expulsion of trapped gas within said needle assembly.

3. The device of claim 2 which further comprises a filter having an inlet in fluid communication with the first lumen and an outlet in fluid communication with the inlet of said tube.

4. The device of claim 1 which further comprises a readily removable cap covering the proximal end of the second compartment.

5. The device of claim 1 wherein said spring is a coil spring that is compressed when said first needle is in the extended position.

6. The device of claim 5 wherein said catheter assembly includes a fitting for readily attaching and releasing intravenous tubing.

7. The device of claim 1 wherein said housing includes an abutment that limits the sliding of said needle assembly to the retracted position, the abutment being at the distal end of the second compartment.

8. The device of claim 1 said catheter assembly including a pair of opposing flexible wings adapted and configured for grasping by a user.

9. The device of claim 1 wherein said tube comprises a flexible material and has a free length that is less than the distance between the extended and retracted positions of said needle assembly, said tube being extended beyond the free length when said needle assembly is in the extended position.

10. The device of claim 1 wherein said tube comprises a flexible material and has a free length that is greater the distance between the extended and retracted positions of said needle assembly, said tube being compressed from the free length and fitting at least partly within the second compartment when said needle assembly is in the extended position.

11. The device of claim 1 which further comprises a fitting on the end of said tube located on the proximal end said second compartment when said needle assembly is in the extended position, said fitting being adapted and configured for readily attaching and releasing the container for collecting blood.

12. The device of claim 11 wherein the fitting includes a second needle adapted and configured for puncturing a seal of the container for collecting blood.

13. The device of claim 12 wherein the sharp tip of said first needle extends out from the distal end of the second lumen when said first needle is in the extended position.

14. A device for collecting blood in a container and connecting to intravenous tubing, comprising:
a housing having a first compartment and a second compartment;
a needle assembly having proximal and distal ends and including a needle defining a first lumen and having a sharp tip on the distal end, said needle assembly being slidable within the first compartment between an extended position in which the sharp tip is external to said housing and a retracted position in which the sharp tip is located within the first compartment;
means for biasing said needle assembly to the retracted position;
a tube in fluid communication with the first lumen and extending from the proximal end of said needle assembly and extending into the second compartment; and
a catheter assembly having a flexible second lumen supported by a body, the second lumen surrounding at least a portion of the needle, said catheter assembly including a pair of opposing flexible wings adapted and configured for grasping by a user.

15. The device of claim 14 which further comprises a gas purging valve that expels trapped gas within said needle assembly.

16. The device of claim 14 which further comprises a filter in fluid communication with the first lumen and providing fluid communication to said tube.

17. The device of claim 14 which further comprises means for retaining said needle assembly in the extended position, said needle assembly automatically moving to the retracted position upon removal of said catheter assembly from said needle assembly.

18. The device of claim 14 which further comprises a readily removable cap covering the proximal end of the second compartment.

19. The device of claim 14 wherein said spring is a coil spring.

20. The device of claim 14 wherein said catheter assembly includes a fitting for attaching intravenous tubing.

21. The device of claim 14 wherein said needle assembly abuts the distal end of the second compartment in the retracted position.

22. The device of claim 14 which further comprises a rigid sheath containing therein said first tube, said rigid sheath being slidable with said needle assembly from the extended position to the retraced position.

23. The device of claim 14 wherein said tube comprises a flexible material and has a free length that is less than the length of the second compartment.

24. The device of claim 14 wherein said tube comprises a flexible material and has a free length that is greater than the length of the second compartment.

25. The device of claim 14 wherein the proximal end of said tube has a fitting adapted and configured for readily attaching and releasing the container for collecting blood.

26. The device of claim 25 wherein said needle is a first needle, and which further comprises a second needle in fluid communication with said tube and adapted and configured for receiving blood from said tube, said second needle being located on the proximal end of said housing.

27. The device of claim 14 wherein said first needle slides linearly along an axis from the extended position to the retracted position.

28. A device for collecting blood in a container and connecting to intravenous tubing, comprising:
a housing having an interior and an abutting surface in the interior;
a needle assembly having proximal and distal ends and including a body supporting a needle defining a first lumen and having a sharp tip on the distal end, said needle assembly being slidable within the interior between an extended position in which the sharp tip is external to said housing and a retracted position in which the proximal end of the body contacts the abutting surface;
a spring biasing said needle assembly to the retracted position;
a tube in fluid communication with the first lumen and extending from the proximal end of said needle assembly, said tube having a proximal end extending generally to the proximal end of the housing, said tube having a fitting adapted and configured for readily attaching and releasing the container for collecting blood; and
a catheter assembly having a second flexible lumen supported by a body, the second lumen surrounding at least a portion of the needle.

29. The device of claim 28 which further comprises a gas purging valve that permits expulsion of trapped gas within said needle assembly and a filter having an inlet in fluid communication with the first lumen and an outlet in fluid communication with the inlet of said tube.

30. The device of claim 28 which further comprises means for retaining said needle assembly in the extended position, said retaining means comprising a friction fit of said needle assembly to said housing, said catheter assembly compressing said retaining means when in the extended position.

31. The device of claim 28 which further comprises a readily removable cap covering the proximal end of the housing, said fitting being attached to said cap.

32. The device of claim 31 wherein the fitting includes a second needle adapted and configured for puncturing a seal of the container for collecting blood, said cap covering said second needle when said needle assembly is in the extended position.

33. The device of claim 32 which further comprises a rigid sheath containing therein said first tube, said rigid sheath being slidable with said needle assembly from the extended position to the retracted position.

34. The device of claim 28 wherein said catheter assembly includes a fitting for readily attaching and releasing intravenous tubing, and a pair of opposing flexible wings adapted and configured for grasping by a user.

35. The device of claim 28 which further comprises a rigid sheath surrounding said tube, the proximal end of said tubing and the proximal end of said rigid sheath extending out of the interior when said needle assembly is in the retracted position.

36. The device of claim 28 wherein said tube comprises a flexible material and has a free length that is less than the length of the interior of said housing, said tube being extended beyond the free length when said needle assembly is in the extended position.

37. The device of claim 28 wherein said tube comprises a flexible material and has a free length that is greater than the length of the second compartment, said tube being compressed from the free length and fitting within the interior of said housing when said needle assembly is in the extended position.

38. The device of claim 28 wherein said needle is a first needle, and which further comprises a second needle in fluid communication with said tube and adapted and configured for receiving blood from said tube.
